(19) United States
(12) Patent Application Publication
(10) Pub. No.: US 2005/0058688 A1
(43) Pub. Date: Mar. 17, 2005

(54) Device for the treatment and prevention of disease, and methods related thereto

(76) Inventors: Lars Boerger, Koln (DE); Wolfgang Daum, Groton, MA (US)

Correspondence Address: Wolfgang Daum, 20 Whiley Road, Groton, MA, 01450, US

(21) Appl. No.: 10/784,331

(22) Filed: Feb. 23, 2004

Related U.S. Application Data
(60) Provisional application No. 60/448,930, filed on Feb. 22, 2003.

(51) Int. Cl. A61F 13/00; C12P 21/06

(52) U.S. Cl. 424/426

(57) ABSTRACT

Disclosed are implantable devices for delivering a drug or pharmaceutical agent into the blood stream of a vessel or into the vessel wall of a human body to treat or prevent vascular or cardiovascular disease, such as vascular plaque, cardiovascular plaque, and diseases attributable to inflammation, such as arteriosclerosis, diabetes, rheumatoid arthritis, and Alzheimer's disease. The devices of the present invention may have a ring-like, flag-like, or plaster-like configuration. Also disclosed are methods related thereto.

### CROSS REFERENCE TO RELATED APPLICATION

This is a non-provisional application claiming the benefit of and priority to U.S. provisional patent application having Ser. No. 60/448,930, as filed on Feb. 22, 2003.

### TECHNICAL FIELD

This present invention relates generally to implantable devices. Specifically, the invention pertains to implantable devices that release a drug or pharmaceutical agent to treat or prevent cardiovascular or vascular diseases, and methods related thereto.

### BACKGROUND

Vascular disease is a leading cause of death and disability. In the United States, more than one half of all deaths are due to cardiovascular disease. Arteriosclerosis is the most common form of vascular disease and leads to insufficient blood supply to body organs, which can result in hearts attacks, strokes, and kidney failure.

1. Atherosclerosis and Plaques

Atherosclerosis is a form of vascular injury in which the vascular smooth muscle cells in the artery wall undergo hyperproliferation and invade and spread into the inner vessel lining, which can make the vessels susceptible to complete blockage when local blood clotting occurs, so called stenosis. This can lead to death of the tissue served by that artery. In the case of a coronary artery, this blockage can lead to myocardial infarction and death. Atherosclerosis (the most common form of arteriosclerosis, marked by cholesterol-lipid-calcium deposits in arterial linings), “hardening” of the arteries caused by plaques and plaque lesions, is the cause of myocardial infarction (MI). These hard plaques are the so-called calcified plaques. However, some plaques are “hard and solid”, and the others are “soft and squishy”. It is the soft variety that is causes the most concern. This soft plaque is also referred to as “vulnerable plaque” because of its tendency to burst or rupture.

Vulnerable plaques are usually those causing only mild to moderate stenosis and having a lipid-rich core and a thin, macrophage-dense, collagen-poor fibrous cap. Factors affecting plaque rupture include mechanical injury, circadian rhythm, inflammation, and infection. Progressive thrombosis and vasospasm may follow plaque rupture. It is believed that physical disruption of such a plaque allows circulating blood coagulation factors to meet with the highly thrombogenic material in the plaque's lipid core, thereby instigating the formation of a potentially occluding and fatal thrombus. Some believe these plaques cause more than 50% cross-sectional stenosis of the artery.

Mechanical stress and composition of plaques play an important role in plaque disruption. Mechanical forces, including the mere vibration of the heart as it beats, can easily disrupt this plaque. These plaques are classified as either “yellow” or “white”, using coronary angioscopy. Yellow plaques with an increased distensibility and a compensatory enlargement may be mechanically and structurally weak. As a result, mechanical “fatigue,” caused by repetitive stretching, may lead to plaque disruption. Plaques with a high distensibility and a compensatory enlargement may be vulnerable. While a rupturing plaque can lead to a heart attack, most of the time nothing much bad happens. In fact, it appears that plaques break or rupture all the time, and those that trigger heart attacks are unfortunate exceptions. It is believed that the large plaques visible on angiograms are often the healed-over and more stable remains of small vulnerable plaques.

One of the most important issues of vulnerable plaque is the fact that vulnerable plaques do not bulge inward. Instead, as a plaque grows, it often protrudes outward, into the wall of the artery, rather than into the channel-lumen where blood flows. On an angiogram, everything can look normal. But when dissected after death, it can be seen that the arteries' walls are thick with plaque which could not yet be seen on an angiogram.

Studies into the composition of vulnerable plaque suggest that the presence of inflammatory cells (and particularly a large lipid core with associated inflammatory cells) is the most powerful predictor of ulceration and/or imminent plaque rupture. In plaque erosion, the endothelium beneath the thrombus is replaced by or interspersed with inflammatory cells.

2. Stents and PTCA

Coronary or any peripheral artery blockage can be treated with artery bypass surgery and/or angioplasty. Both procedures may initially appear to be successful, but are in effect undone by the effect of restenosis or the recurrence of stenosis after such a treatment. Restenosis is believed to include hyperproliferation of vascular smooth muscle cells. In particular, one third of patients treated using angioplasty have restenosis and blockage within 6 months after the procedure. To prevent vessel blockage from restenosis, stents are used.

Known stent designs include monofilament wire coil stents (e.g., U.S. Pat. No. 4,969,458); welded metal cages (e.g., U.S. Pat. No. 4,733,665 and U.S. Pat. No. 4,776,337); and, most prominently, thin-walled metal cylinders with axial slots formed around the circumference (e.g., U.S. Pat. No. 4,733,665; U.S. Pat. No. 4,739,762; and U.S. Pat. No. 4,776,337). Known construction materials for use in stents include polymers, organic fabrics and biocompatible metals, such as, stainless steel, gold, silver, tantalum, titanium, and shape memory alloys such as Nitinol.

Of the many problems that may be addressed through stent-based local delivery of beneficial agents, one of the most important is restenosis. Restenosis is a major complication that can arise following vascular interventions such as angioplasty and the implantation of stents. Simply defined, restenosis is a wound healing process that reduces the vessel lumen diameter by extracellular matrix deposition and vascular smooth muscle cell proliferation, and which may ultimately result in renarrowing or even reocclusion of the lumen. Despite the introduction of improved surgical techniques, devices and pharmaceutical agents, the overall restenosis rate is still reported in the range of 25% to 50% within six to twelve months after an angioplasty procedure. To treat this condition, additional revascularization procedures are frequently required, thereby increasing trauma and risk to the patient, as well as increasing the costs of health care.

Some of the techniques under development to address the problem of restenosis include irradiation of the injury site and the use of conventional stents to deliver a variety of beneficial or pharmaceutical agents to the wall of the traumatized vessel. In the latter case, a conventional stent is frequently surface-coated with a beneficial agent (often a drug-impregnated polymer) and implanted at the angioplasty site. Alternatively, an external drug-impregnated polymer sheath is mounted over the stent and co-deployed in the vessel.

Percutaneous transluminal coronary angioplasty (PTCA) is used as the primary treatment modality in many patients with coronary artery disease. PTCA can relieve myocardial ischemia in patients with coronary artery disease by reducing lumen obstruction and improving coronary flow. Stents and PTCA balloon catheters are usually used for the hard and calcified plaques. There are no solutions on the market yet to treat or prevent the soft or vulnerable plaques.

3. Taxol®

Therapeutic agents to inhibit restenosis have been used with varying success. Taxol®, an antimicrotubule agent isolated from the bark of the western Pacific Yew tree, is especially effective in inhibiting some cancers and is shown to be effective in combating restenosis (e.g., U.S. Pat. No. 5,733,925). Taxol® may also prevent thrombus formation. Because systemic administration of Taxol® can have undesirable side effects, local administration is a preferred mode of treatment.

At least five considerations appear, on their face, to preclude the use of inhibitory drugs to prevent stenosis resulting from overgrowth of smooth muscle cells.

A. Inhibitory agents may have systemic toxicity that could create an unacceptable level of risk for patients with cardiovascular disease.

B. Inhibitory agents may interfere with vascular wound healing following surgery and that could either delay healing or weaken the structure or elasticity of the newly healed vessel wall.

C. Inhibitory agents killing smooth muscle cells could damage the surrounding endothelium and/or other medial smooth muscle cells. Dead and dying cells also release mitogenic agents that might stimulate additional smooth muscle cell proliferation and exacerbate stenosis.

D. Delivery of therapeutically effective levels of an inhibitory agent may be problematic from several standpoints: namely,
a. delivery of a large number of molecules into the intercellular spaces between smooth muscle cells may be necessary, i.e., to establish favorable conditions for allowing a therapeutically effective dose of molecules to cross the cell membrane;
b. directing an inhibitory drug into the proper intracellular compartment, i.e., where its action is exerted, may be difficult to control; and,
c. optimizing the association of the inhibitory drug with its intracellular target, e.g., a ribosome, while minimizing intercellular redistribution of the drug, e.g., to neighboring cells, may be difficult.

E. Because smooth muscle cell proliferation takes place over several weeks, it would appear a priori that the inhibitory drugs should also be administered over several weeks, perhaps continuously, to produce a beneficial effect.

Hence, local administration of Taxol® may be more effective when carried out over a longer time period, such as a time period at least matching the normal reaction time of the body to the angioplasty. Local administration of Taxol® over a period of days or even months may be most effective in inhibiting restenosis. Such a long time period may be successfully provided by a time-release delivery system utilizing a Taxol® coated stent. There are different derivatives of Taxol®, such as Paclitaxel™.

It has been demonstrated that Paclitaxel™ polymer-coated stents reduce neointima formation (see, Farb A, Heller P, Shroff S, Cheng L, Kolodgie F, Carter A, Scott D, Froehlich J, Virmani R, “Pathological Analysis of Local Delivery of Paclitaxel Via a Polymer-Coated Stent”, Circulation. 2001; 104:473).

4. Biodegradable Materials

There are many biodegradable polymers in the market, that can be used, and including those that have proper biomedical approval for use in humans. Basic biodegradable polymers include starch, cellulose, amylose, polyhydroxybutyrate, lactic or polyactic acid, polybuylenesuccinate, polycaprolactone, aliphatic-aromatic resin, carboxymethylcellulose (CMC) or thermal polyasparatate (TPA).

It has been long known that polylactides comprising poly(L-lactide), poly(D-lactide) or copolymers derived therefrom or with other comonomers in the form of copolymerizable cyclic esters are usable for human implantable devices.

More recently, several bioabsorbable, biocompatible polymers have been developed for use in medical devices, and approved for such use by the U.S. Food and Drug Administration (FDA). These FDA approved materials include polyglycolic acid (PGA), polylactic acid (PLA), Polyglactin 910 (comprising a 9:1 ratio of glycolide per lactide unit, and known also as VICRYL™), polyglyconate (comprising a 9:1 ratio of glycolide per trimethylene carbonate unit, and known also as MAXON™), and polydioxanone (PDS). In general, these materials biodegrade in vivo in a matter of months, although certain more crystalline forms biodegrade more slowly. These materials have been used in orthopedic applications, wound healing applications, and extensively in sutures after processing into fibers. Some of these polymers also have been used in tissue engineering applications.

It has been reported that the polymer hydtoxyethyl methacrylate-vinyl pirrolidone is biodegradable and lacks toxicity toward the cells and hence is capable of being used for local drug delivery systems (see, Gimeno M J, Garcia-Esteo F, Garcia-Honduvilla N, Bellon J M, Bujan J, Roman J S, Polymer controlled drug delivery system for growth hormone, Drug Deliv 2002 October-December; 9(4):233-7).

5. Local Drug Delivery Systems

Newly designed metallic stent containing honeycombed strut elements with inlaid stacked layers of Paclitaxel™ and biodegradable polymer has been demonstrated for instent restenosis prevention (see, Finkelstein et. al. “Local Drug Delivery via a Coronary Stent With Programmable Release Pharmacokinetics”, Circulation. 2003; 107:777). In an in vitro study it was shown that manipulation of the layers of biodegradable polymer and drug allowed varying of the initial 24-hour burst release of Paclitaxel™ from 69% to 8.6%. Late release of drug could be adjusted dependently or independently of early burst release. A biphasic release profile was created by the addition of blank layers of polymer within the stack. In the 30-day porcine coronary model, there was a 70% reduction in late loss, a 28% increase in luminal volume, and a 50% decrease in histological neointimal area compared with bare metal controls. The disadvantage of this approach is that the stent remains in the body after the drug and the bioresorbable coating vanish, even in the case where a stent is no longer necessary.

A useful means for attaching an effective drug to a bioabsorbable carrier reportedly involves a pharmaceutical composition in the form of a solid carrier consisting of a substrate and an encapsulation coat on the substrate, wherein the encapsulation coat comprises an admixture of a therapeutically effective amount of a hydrophilic pharmaceutical active ingredient, an effective solubilizing amount, and a lipophilic additive selected from the group consisting of lipophilic surfactants, triglycerides, and combinations thereof, and wherein the effective solubilizing amount solubilizes the pharmaceutical active ingredient in the encapsulation coat (e.g., U.S. Pat. No. 6,248,363). However, this approach does not provide such bioresorbable material to be modified for a medical implantable device.

Hydrogel-forming polymeric materials have been found to be useful in the formulation of medical devices, such as drug delivery devices. Hydrogel-forming polymers are polymers that are capable of absorbing a substantial amount of water to form elastic or inelastic gels. Many non-toxic hydrogel-forming polymers are known and are easy to formulate. Medical devices incorporating hydrogel-forming polymers offer the flexibility of being capable of implantable in liquid or gelled form. Once implanted, the hydrogel-forming polymer absorbs water and thus swells. The release of a pharmacologically active agent incorporated into the device takes place through this gelled matrix via a diffusion mechanism. However, many hydrogels, although biocompatible, are not biodegradable or are not capable of being formed to stable solid devices, which then also dissolve over time.

A list of suitable mechanical properties for biocompatible polymers for tissue engineering and lists typical devices for applications are disclosed in U.S. Pat. No. 6,514,515. However, this patent does not teach how the listed devices are designed or able to carry drugs and dissolve completely over time.

Other reported approaches for delivery of drugs or pharmaceutical agents include:
Parenteral delivery of a drug in a biodegradable polymeric matrix to a warm blooded animal, wherein the polymeric matrix comprises a member selected from the group consisting of poly(α-hydroxy acids) and poly(ethylene carbonates) (e.g., U.S. Pat. No. 5,702,717). The drug is released at a controlled rate from the copolymer, which biodegrades into non-toxic products. The degradation rate can be adjusted by proper selection of the poly(α-hydroxy acid).
A transdermal therapeutic system (TTS) for the transcutaneous administration of pergolide over several days (e.g., U.S. Pat. No. 6,461,636). The TTS contains a matrix mass, containing pergolide, taking the form of a layer, which contains a (meth)acrylate copolymer containing ammonio groups or a mixture of a (meth)acrylate copolymer containing amino groups and a (meth)acrylate polymer containing carboxyl groups, 10-50% by weight propylene glycol and up to 5% by weight pergolide.
An expandable medical device, which is stent-like and which has a plurality of elongated struts (e.g., US 20020082680). Some of the stent struts include openings in which drugs are integrated for release over time. As with other drug eluting stent embodiments, the stent in this case remains in the body after the drugs have been released, even when the drugs have dispersed and the disease may have been cured.
A device comprising an ocular implant which bio-erodes within the eye environment thereby gradually releasing the therapeutic agents at the site to be treated until the entire implant eventually erodes without the need for further surgery (e.g., U.S. Pat. No. 4,863,457). Unfortunately, the particular polymer used is not identified. This device does not apply to cardiovascular diseases.
An erodeble device for delivering a drug into the human body, comprising a poly(orthoester) or a poly(orthocarbonate) (e.g., U.S. Pat. No. 4,346,709). It is unclear if the device is intended to erode completely or only partially. No drug that is useful for any vascular disease, nor the use of the device for vascular diseases, are disclosed.
A new protein matrix material for implantable medical devices and implantable drug delivering devices and methods of making such materials (e.g., US 20020028243). Although the use of this protein based material, which totally disperses, is mentioned for the use of implants, only examples given are those of encapsulated or coated stents, in which only the coating vanishes. This protein based material appears well-suited for growth of cells on and/or within the material matrix, but it is not suitable for rigid implants due to the fragile nature of the protein.

### SUMMARY OF THE INVENTION

In view of the above, there is a need for an implantable device that releases a drug over a period of time, and dissolves or degrades thereafter. There is also a need for methods related to such devices for the treatment or prevention of cardiovascular or vascular diseases.

It is, therefore, an aspect of the present invention to provide an implantable device that releases one or more drugs, preferably over a period of time, and dissolves thereafter.

It is also an aspect of the present invention to provide an implantable device for the treatment or prevention of cardiovascular or vascular diseases.

The present invention pertains to an implantable device comprising a biodegradable material, which is coated, loaded or filled with a drug or pharmaceutical agent that releases over a period of time, for the treatment or prevention of cardiovascular or vascular diseases, diseases resulting from inflammation, and hard, soft, calcified or vulnerable plaque. The present invention also pertains to methods related to such devices.

The devices of the present invention is also suitable for use with patients who have already undergone vascular procedures, for instance a PTCA, or who are classified as high-risk patients due to their family history, their high LDL (low density lipoprotein) or CRP (C-Reactive Protein) levels. The device as disclosed herein is useful for local delivery of drugs or pharmaceutical agents to treat coronary disease, such as plaques or stenosis. This device may also be used as an alternative to stents, for patients who comprise multiple stents in a treated vessel.

The devices of the present invention may embody any of various structures, particularly a ring-like structure, a flag-like structure, and a plaster-like structure.

The ring-like structure (RLS) is deployed in a vessel and fixed to a defined position by gently pushing it outwards against the vessel wall. The RLS comprises a biodegradable matrix material, in which a drug is incorporated and released over time. Alternatively, the RLS may comprise a biodegradable matrix coated with a drug or a drug-containing polymer from which the drug dissolves over time. The RLS may also comprise a drug releasing substance. Preferably the drug, as well as the drug releasing substance, and the biodegradable matrix dissolve and vanish over time. The RLS may have a circular, elliptical, or any other configuration having circular geometry. The flag-like structure (FLS) is deployed in a vessel and hangs on a ring, which itself is fixed to a defined position in the vessel by as it is gently pushed outwards against the vessel wall. The FLS comprises a biodegradable matrix material and a drug which is released over time. The FLS may also comprise a drug releasing substance. The drug, as well as the drug releasing substance, and the biodegradable matrix dissolve and vanish over time. Compared to the RLS, the FLS comprises a larger drug-eluting surface and hence releases the drug more quickly.

The plaster-like structure (PLS) is deployed on the vessel wall. The PLS comprises a biodegradable matrix material and a drug which is released over time. The PLS may also comprise a drug releasing substance. The drug, as well as the drug releasing substance, and the biodegradable matrix dissolve and vanish over time.

The above summary of the present invention is not intended to describe each illustrated embodiment or every implementation of the present invention. The figures and the detailed description that follow particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 schematically illustrates an implantable device, in the form of a ring-like structure, deployed in a manner similar to that of a stent, according to one embodiment of the present invention:

FIG. 1a illustrates a system for deploying the implantable device,

FIGS. 1b, 1d, and 1f illustrate longitudinal sectional views of the implantable device,

FIGS. 1c, 1e, and 1g illustrate cross sectional views of the implantable device;

FIG. 2 schematically illustrates a form of the ring-like structure, according to one embodiment of the present invention:

FIG. 2a illustrates a three-dimensional view of FIG. 1a, and

FIG. 2b illustrates an unfolded view of FIG. 1b;

FIG. 3 schematically illustrates a cross sectional view of a ring-like structure strap having a coating on the inner side, according to one embodiment of the present invention;

FIG. 4 schematically illustrates a method for deploying an implantable device, in the form of a ring-like structure, according to one embodiment of the present invention,

FIG. 4a illustrates a system for deploying the implantable device,

FIG. 4b illustrates the system with a balloon in expanded mode,

FIG. 4c illustrates the system with a balloon in contracted mode,

FIGS. 4d and 4e illustrate the implantable device mounted on an expanded spring;

FIG. 5 schematically illustrates an implantable device, in the form of a flag-like structure, in a coronary vessel, behind the location where the vessel branches from the ascending aorta, according to one embodiment of the present invention;

FIG. 6 schematically illustrates an implantable device, in the form of a flag-like structure, which is constructed from tapered fibers, according to one embodiment of the present invention,

FIG. 6a illustrates a cross sectional view of woven or twisted fibers,

FIG. 6b illustrates a cross sectional view of floating unwoven fibers,

FIG. 6c illustrates a three dimensional view of a tapered fiber;

FIG. 7 schematically illustrates an implantable device, in the form of a plaster-like structure, which is deployed in a manner similar to that of a stent, according to one embodiment of the present invention:

FIG. 7a, illustrates a system for deploying the implantable device,

FIGS. 7b, 7d, and 7f illustrate longitudinal sectional views of the implantable device,

FIGS. 7c, 7e, and 7g illustrate cross sectional views of the implantable device; and,

FIG. 8 schematically illustrates a cross sectional view of the material matrix of an implantable device comprising coated particles, according to one embodiment of the present invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION OF EXAMPLARY EMBODIMENTS

The present invention pertains to implantable devices for delivery of a drug into a vessel system of a patient's body, e.g., the cardiovascular or coronary system, to treat the vessel system or parts of the vessel system, or prevent the vessel system or part of the vessel system from a disease. The drug may be coated onto one or more surfaces or incorporated into the matrix material, or coated onto fibers that are incorporated into the matrix material of the drug-delivering implantable device. The drug is released from the device over a period of time, with the rate of release being based on body temperature and chemical, biochemical, or physical reactions between the device and the blood. Thereafter, the device dissolves, and is preferably removed from the body by the body's natural processes.

1. Definitions

The terms “elution” or “elute”, “diffusion” or “diffuses”, “dissolve”, and “controlled release” or “releases” as used herein, refer to a process in which a drug leaves the drug-containing matrix. The physical, chemical or biochemical process between elution, diffusion, and dissolving may be different.

The terms “diffusion” or “diffuses”, “degredation” or “degrades”, “dissolves”, and “erosion” or “erodes”, as used herein, refer to a process in which matrix material leaves body. The physical, chemical or biochemical process between diffusion, degradation, dissolving, and erosion may be different.

The term “matrix”, as used herein, refers to the material environment of the device and describes a material composition of different materials, elements, and etc., or combinations of materials, drugs and particles. The matrix comprises a drug or fibers coated with a drug.

The terms “biodegradable”, “bioabsorbable”, and “bioresorbable”, as used herein, refer to any material that is in contact with human body tissue or fluids and that is susceptible to breakdown to lesser molecular weight components.

The terms “drug”, “pharmacologically active agent”, and “pharmaceutical agent” and “pharmaceutical composition”, as used herein, refer to a substance or medication used in the diagnosis, treatment, or prevention of a disease.

The term “plaque”, as used herein, refers to calcified vascular plaque, vulnerable vascular plaque, hard vascular plaque, soft vascular plaque, or any combination of these.

The term “to deploy a device into a vessel”, as used herein, refers to determining a beneficial location for implanting the device, preferably using any type of radiological imaging modality, introducing the device into the vessel, and placing the device therein, and leaving it at the beneficial location.

It is to be understood that the singular forms of “a”, “an”, and “the”, as used herein and in the appended claims, include plural reference unless the context clearly dictates otherwise.

2. Implantable Device Configurations

The implantable devices of the present invention embody any of various structures, particularly a ring-like structure, a flag-like structure, and a plaster-like structure.

A. Ring-Like Structure (RLS)

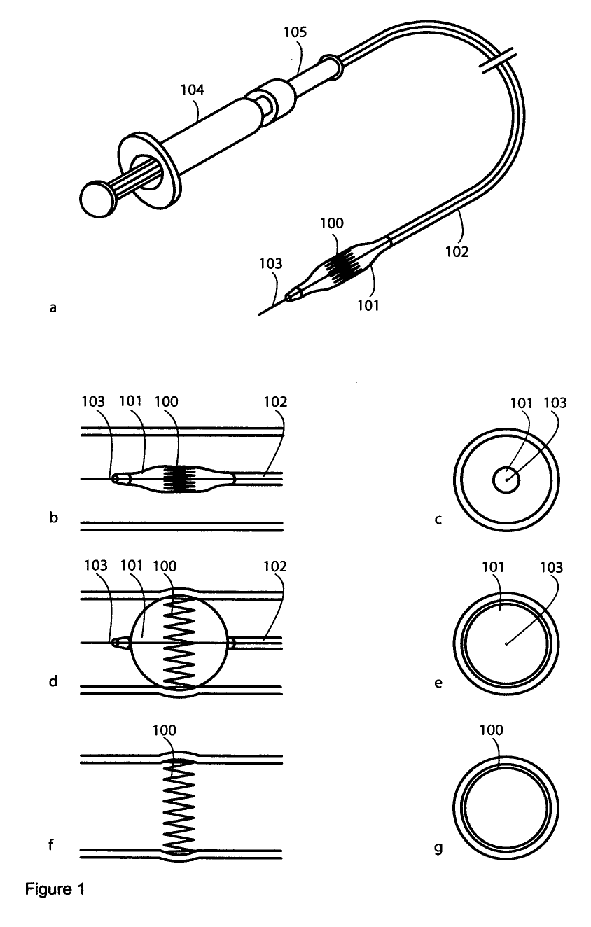

A ring-like structure (RLS), which can be deployed in a manner similar to a standard state of the art vascular or cardiovascular stent, is demonstrated in FIG. 1. An RLS 100 is mounted on a balloon 101, in a manner similar to mounting a stent, and the balloon 101 is mounted on a catheter system 102, as illustrated in FIG. 1a. The catheter system 102 with balloon 101 is guided with the help of a guide wire 103 into a vessel 106. A syringe 104 is adapted via an adapter 105 onto the catheter system 102 to expand the balloon 101, e.g., with NaCl (sodium-chloride) solution. This procedure is illustrated in FIG. 1b through FIG. 1g. The balloon 101 with RLS 100 is guided with the guide wire 103 in the vessel 106, as illustrated in FIG. 1b and FIG. 1c, under standard imaging modalities, such as ionising radiation (x-ray), ultrasound (US) or magnetic resonance imaging (MRI). The balloon 101 is then expanded, as illustrated in FIG. 1d and FIG. 1e, and the RLS 100 stretches beyond its elastic limits, thus becoming deformed. Once the balloon 101 is contracted and removed, the RLS 100 will remain in the vessel 106, gently clamping itself outwards against the wall of vessel 106, and thus remaining at that position, as illustrated in FIG. 1f and FIG. 1g.

An implantable device having a ring-like structure (RLS) differs from a stent in that the stent requires more mechanical strength to push against the vessel wall as the stent has to hold open the vessel, while the RLS only has to clamp itself against the vessel wall to stay in place. The purpose of a stent is to hold the vessel open by mechanical strength to prevent the vessel from occlusion, while the purpose of the RLS is to stay in place and release a drug. Although the RLS may be as long as a stent or shaped exactly like a stent, the two devices serve different purposes. Because a stent in general has to hold the vessel open over a longer distance of space and a longer duration of time, the stent must be constructed in such a way that it sustains more mechanical force. Because the RLS has to carry a drug to be released into the blood stream or to the vessel wall itself, and to dissolve and vanish thereafter, it does not need to be constructed to sustain a mechanical force of the magnitude required for a stent. Although some stents may comprise drug coatings, the purpose of those coatings is to prevent the stent from re-closing or re-occluding, referred to as instent-restenosis. Whereas the drug of an RLS is effective in the blood itself, on the vessel's inner surface, in the vessel wall further down the blood stream, or in the vessel wall at the location of the RLS.

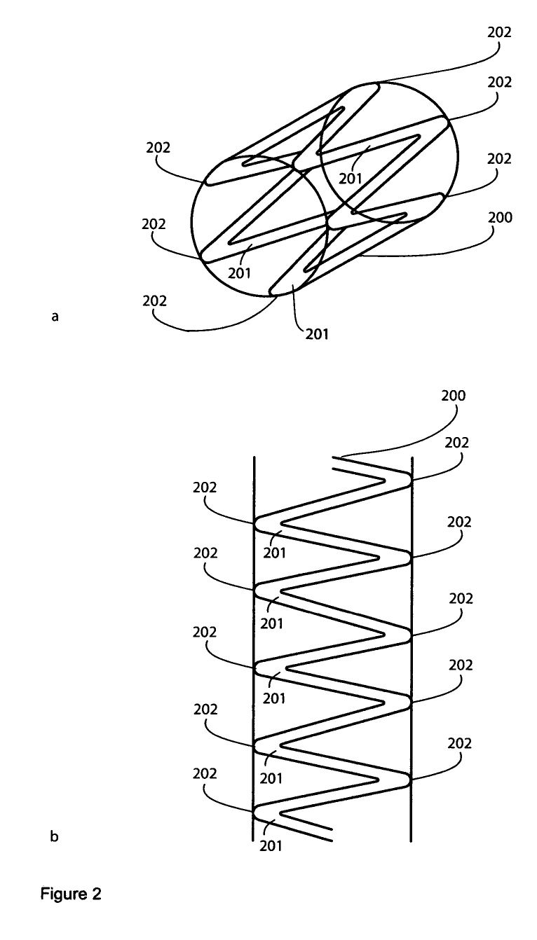

In one embodiment of the present invention, the RLS comprises a zigzag or weave structure, as illustrated in FIG. 1 and illustrated in three-dimension in FIG. 2a. An RLS structure 200 is cut from a tube using a laser, in a manner similar to standard stents. Alternatively, the RLS may be cut from a sheet and glued or welded to yield a tube-like geometry, as illustrated in FIG. 2a. The RLS 200 as unfolded is illustrated in FIG. 2b. During the expansion of the RLS 200, the straps 201 at the edges 202 bend over their elastic limits and become plastically deformed to remain in the expanded geometry. The straps are pieces of the RLS that are deformed beyond its plasticity limits so that the RLS does not bend back. The RLS may comprise one or more straps. Different geometries and designs are also possible for the RLS.

The RLS is preferably constructed from a biodegradable matrix material, one that dissolves or degrades over time, more preferably one that dissolves or degrades after a drug coating has dissolved or after the drug incorporated in the matrix has washed out, diffused out, dissolved, or eluted.

In one embodiment of the present invention, the RLS is deployed e.g., in a cardiovascular artery or any other vessel proximal to or at the area in which the drug shall be effective. To treat an entire artery, the RLS may be deployed in the artery behind the location where the artery branches from the ascending aorta. The RLS typically has the following dimensions:
unexpanded diameter: about 0.5 to about 5 mm, preferably 1-2 mm,
expanded diameter: about 2 to about 12 mm, preferably 3-5 mm,
wall thickness: about 0.07 to about 0.5 mm, preferably 0.07-0.12 mm,
length: about 3 to about 20 mm, preferably 4-6 mm,
strap-width: about 0.1 to about 5 mm, preferably 0.1-1 mm.

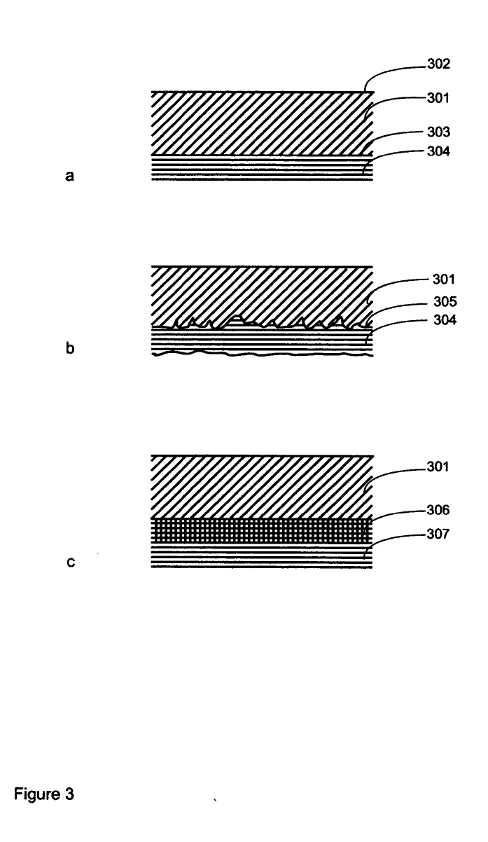

The RLS has basically two surfaces, the outer surface facing the vessel wall and the inner surface facing the lumen or the blood stream in the vessel. In one embodiment of the present invention, at least the inner surface of the RLS is coated with a drug. The drug is released by the coating and it dissolves in the blood stream, whereby it is transported downstream the vessel to be effective on sites of the vessel wall or in further vessels distal to the location in which the RLS was deployed. FIG. 3a illustrates a cross sectional view of a strap 301 of an RLS, having an outer surface 302, an inner surface 303 and a drug containing layer 304, which is coated on the inner surface 303. In one embodiment of the invention, the drug-containing layer is coated on the outer surface 302. The drug that is coated on the outer surface 302 and the drug that is coated on the inner surface 303 may be the same drug or it may be different.

Un-isotropic chemical or physical etching may increase the roughness of the inner surface 305 of the strap 301 of an RLS, as illustrated in FIG. 3b. This type of etching selectively etches grain-boundaries in the material, giving the surface a larger surface for the coating. There may be more than one drug-containing surface. In FIG. 3c, two drug-containing coatings 306 and 307 are illustrated. Coating 307 may dissolve before coating 306 dissolves. Coating 307 may dissolve more quickly than coating 306. For example, the purpose of coating 307 may be to treat an acute or severe disease, such as the beginning of a stenosis or vulnerable plaque, while the purpose of coating 306 may be to prevent the vessel from the recurrence of said disease in the near future.

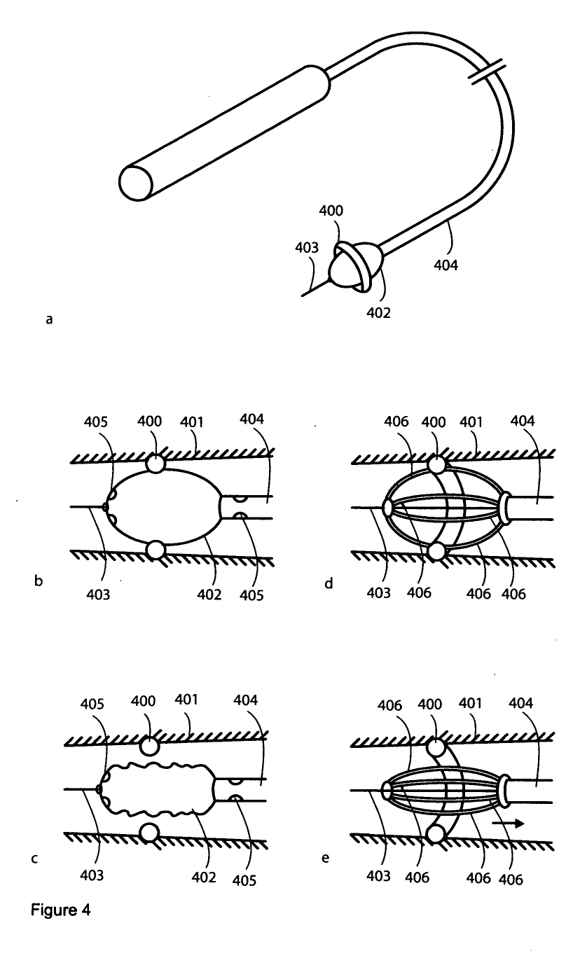

Another method for deploying an RLS in a coronary vessel is illustrated in FIG. 4. A device 400, having a ring-like structure and comprising a ring, is introduced into the vascular system from the external iliac artery, passing into the aorta upstream. The device 400 is then pushed downstream in a desired cardiovascular vessel 401, thus the diameter of the vessel 401 decreases. The intent for this device is to enter the vascular system with a ring in its full dimensions and push it down the cardiovascular artery until the RLS 400 can not be pushed any further and the diameter of the RLS ring is the same as that of the artery and, whereby the RLS comes to a stop. At this position, the RLS 400 clamps itself into the cross section of the artery. The introduction of a full size ring having an outer diameter of about 3 mm to about 4 mm into the iliac artery or the aorta is not shown herein, but may be accomplished, e.g., with a needle of appropriate inner diameter. RLS 400 is mounted on an expanded balloon 402 and is pushed through the vessel with the help of a guide wire 403 and catheter 404. FIG. 4a and FIG. 4b illustrate the process in a longitudinal cross sectional view. In FIG. 4a, balloon 402 is mounted on the distal part of catheter 404, in an expanded mode. Pass through holes 405 allow blood to flow from the proximal side of the balloon 402 to the distal side of the balloon 402. Once the RLS 400 is clamped into the wall of vessel 401, the balloon 402 is deflated and can be withdrawn from the site, as illustrated in FIG. 4c. The RLS 400 is thus deployed.

The use of a balloon is disadvantageous in that blood cannot flow steadily during the time of deployment. FIG. 4d and FIG. 4e illustrate an enhanced system in which the RLS 400 is mounted on an expanded spring 406. When the RLS 400 is clamped in the wall of vessel 401, the spring 406 is released by pulling back the catheter 404, as shown with the arrow in FIG. 4e, and the catheter 404 is withdrawn from the vessel.

A biodegradable RLS may comprise one or more areas or layers of material that degrade, dissolve, elute or vanish over time. These areas or layers may be different and may comprise different drugs, or comprise the same drug but in differing concentrations.

The RLS may have a circular, elliptical, or any other configuration having circular geometry.

B. Flag-Like Structure (FLS)

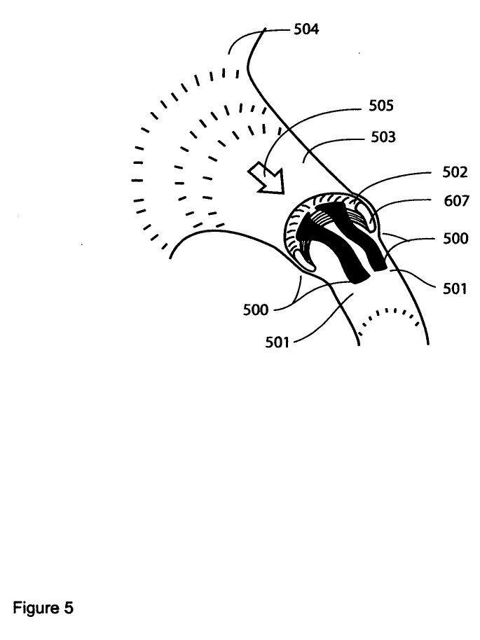

FIG. 5 illustrates a flag-like structure FLS 500. Fibers 501 that comprise a drug are attached to a holding structure 502, which gently clamps itself from inside out against the wall of a coronary vessel 503, behind the location where the vessel branches from an ascending aorta 504. The fibers 501 are elastic and float in the blood stream, as indicated by arrow 505. The holding structure 502 may be a stent or a ring structure, which is deployed in a manner similar to that of a balloon, an expandable stent, or an RLS, as described above.

The FLS may be constructed from fibers, woven tissue, strings or sheets. The matrix material is preferably a biodegradable polymer, more preferably, the polymeric matrix comprises a drug. A drug may also be coated onto or underneath the matrix material.

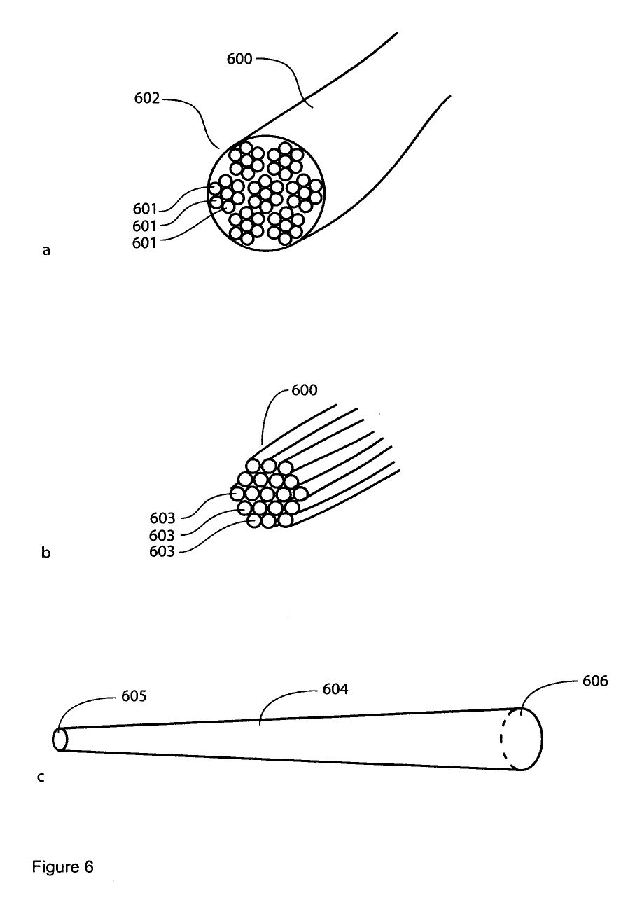

In FIG. 6a, the structure of an FLS 600 is illustrated where single fibers 601 form a substructure 602, which then can be woven or twisted to yield the overall structure. The fibers 603 may also be unwoven, and lie or float in the bloodstream while remaining attached to substructure 602, as illustrated in FIG. 6b. A fiber 604 may be tapered, with a thinner distal portion 605 as compared to a proximal portion 606, as illustrated in FIG. 6c. If such a taping fiber biologically degrades over time, it will diminish from the distal portion 605, leaving the proximal portion 606 attached to a holding structure 607. Hence, there will be no broken-fiber parts drifting apart from the FLS 600, leaving the principle structure intact, which would happen, if the cross section of the fibers would stay constant over the distance along the longitudinal axis of the fiber.

The simplest way to coat the fibers with the drug is to dip them at least once into the drug. The fibers have the advantage of having a high ratio of surface to volume, referred to as aspect ratio, which enables the fibers to be coated with a large amount of drug. Alternatively, the drug may be mixed into the biodegradable polymer matrix and it would elute as the matrix material of the fibers degrade, and thereby extending the effective lifetime of the drug.

The fibers may comprise the same drug or different drugs. Some fibers may comprise drugs that elute more quickly than others, or that have a different effect. Different fibers may comprise different drugs to treat different diseases or different aspects of a disease. For example, some fibers may contain a drug that treats calcified plaque and others may contain a drug that treats vulnerable plaque.

C. Plaster-Like Structure (PLS)

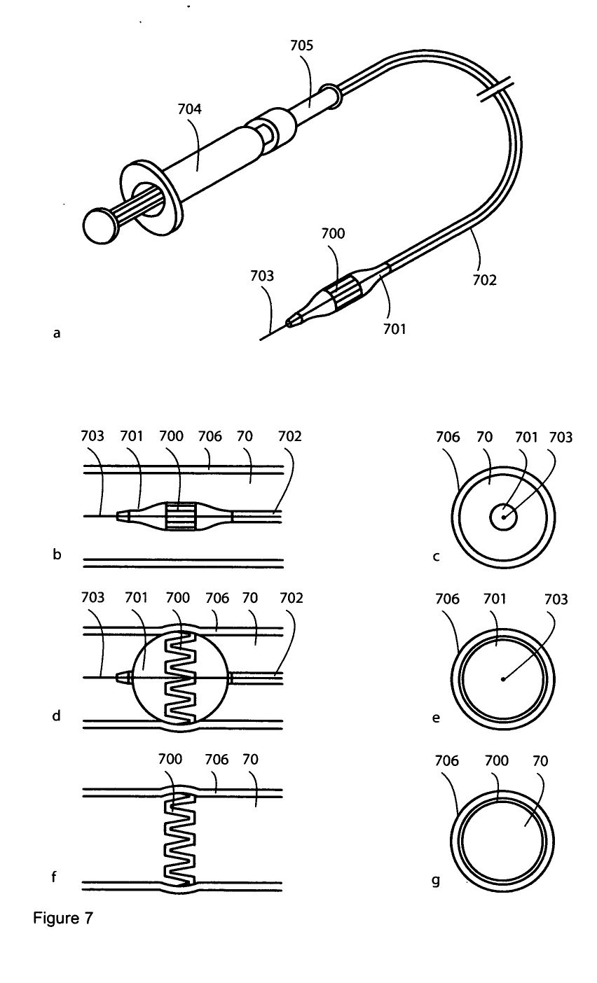

FIG. 7 demonstrates a plaster like structure PLS 700, which can be deployed in a manner similar to a standard state of the art vascular or cardiovascular stent. The plaster material of the PLS comprises a glue to facilitate attachment to the vessel wall. The PLS 700 is mounted on a balloon 701, in a manner similar to a standard stent, with the balloon 701 being mounted on a catheter system 702. The catheter system 702 with balloon 701 is guided with the help of a guide wire 703 into and through a vessel 706. A syringe 704 is adapted via an adapter 705 onto the catheter system 702 to expand the balloon 701, e.g., with NaCl (sodium-chloride) solution. This procedure is illustrated in FIG. 7b through FIG. 7g. The balloon 701 with PLS 700 is guided with the guide wire 703 in the vessel 704, as illustrated in FIG. 7b and FIG. 7c. The balloon 701 is then expanded, as illustrated in FIG. 7d and FIG. 7e, whereby the PLS 700 stretches and sticks, via the glue, to the wall of vessel 706. The glue does not stick below a threshold temperature, e.g., 40° C., and will melt and stick above said threshold temperature. Once the balloon 701 is contracted by releasing the pressure from the balloon, the PLS 700 remains at the wall of vessel 706 in that position, as illustrated in FIG. 7f and FIG. 7g.

In another embodiment of the invention, the matrix material of the PLS is an elastic material and will harden, once the temperature of the NaCl solution in the balloon is raised above a defined threshold temperature. It must be noted that the RLS, as well as the ring of the FLS, may be deployed in the same manner, via hardening a material by temperature change. To reach the threshold temperature in the NaCl solution, an energy source heating, e.g., a heating means or heating element, may be used within the balloon, or a warmed NaCl solution may be pumped into the balloon excorporeally. The energy source may be a resistive electrical wire, a laser (such as a diode laser) or laser fiber, a radio frequency or microwave source, or a chemical reaction.

The plaster-like structure is constructed from a biodegradable polymer, and hence will dissolve over time. Even in the late phase of the bioresorption of the plaster, no parts loosen from the vessel wall to drift into the blood stream and to lead to an occlusion of the vessel because any remaining unresorbed fragments typically remain glued to the vessel wall. This is an advantage of the PLS technique. The PLS may comprise one or more areas or layers, which may be different, and which comprise different drugs, or the same drug but in differing concentrations.

3. Device Composition

In one embodiment of the present invention, the device comprises a biodegradable matrix and a drug.

In one embodiment of the present invention, the biodegradable matrix comprises drug-coated particles. Because not every drug can easily be mixed into the polymer, it may be more efficient to coat the drug on particles and mix these coated particles into the polymer matrix. The particles may comprise the same polymer that the polymer matrix comprises, or they may be selected from different materials, such as iron-oxide (Fe₃O₄), titanium, titaniumalloys, titaniumoxide (TiO₂), manganese oxide, magnesiumoxide, palladiumoxide, or palladiumcobalt. Each particle may also be coated with a binding-layer, which binds the drug to the particle. Such a binding coating may comprise dextran, any sugar based substance, starch, chitosan, agarose or albumin. In one embodiment of the present invention, particles are coated with synthetic polymers, such as poly(lactic acid), poly(ethylene imine), or poly(alkylcyanoacrylate). Typical particle size ranges from about 40 nanometers to about 1 micrometer, preferably from about 100 nanometers to about 400 nanometers. The smaller the particle, the better it will be “digested” or removed by the body's metabolism. The thickness of a typical binding layer is from about 1 nanometer to about 20 nanometers.

Other materials that may be incorporated into the matrix which are not considered polymers, but provide enhanced features include, but are not limited to, ceramics, bioceramics, glasses bioglasses, glass-ceramics, resin cement, resin fill; more specifically, glass ionomer, hydroxyapatite, calcium sulfate, Al₂O₃, tricalcium phosphate, calcium phosphate salts, alginate, carbon, and alloys, such as cobalt-based, galvanic-based, stainless steel-based, titanium-based, zirconium oxide, zirconia, aluminum-based, vanadium-based, molybdenum-based, nickel-based, iron-based, and zinc-based alloys (e.g., zinc phosphate, and zinc polycarboxylate).

In one embodiment of the present invention, the particles are selected to change the contrast in a radiologic imaging system, such as x-ray (fluoroscopy, angiography, CT, etc.), magnetic resonance imaging (MRI), ultrasound (US) or gamma imaging, such as positron emission tomography (PET). For example, Fe₃O₄ changes the magnetic field around itself and hence lowers the T₁ and T₂ signals in MRI and is mostly seen as black spot. Fe₃O₄ also absorbs x-rays and changes the contrast in x-ray based techniques. Radioactive isotopes, such as ⁹⁰Y, ¹³³Xe, ^{81m}Kr, ¹¹¹In, ^{133m}In, or ²⁰¹Th may be inserted into the mixture to render the device imageable under radioactivity detectors. Gd-DTPA contrast media or gadolinium ions may be inserted into the mixture to render the device MR visible; barium contrast media or barium ions would render the device x-ray visible, and small bubble filled with CO₂ would render the device visible for ultrasound.

One advantage of using a polymer-particle composition for constructing an implantable device of the present invention is that this technique allows the use of polymers, proteins, elastins, or collagens. Typically, these materials, due to their mechanical instability or fast dilution characteristic, are not able to form a solid device with long lasting dilution characteristics. In the devices of the present invention, the polymers, proteins or collagens form the binding network between the drug-coated particles. A biocompatible protein for use herein may be naturally occurring, synthetic or genetically engineered. Naturally occurring proteins include, but are not limited to elastin, collagen, albumin, keratin, fibronectin, silk, silk fibroin, actin, myosin, fibrinogen, thrombin, aprotinin, antithrombin III, and any other biocompatible natural protein. Specific examples of a particularly preferred genetically engineered proteins for use in the devices of the present invention include those commercially available under the nomenclature “ELP”, “SLP”, “CLP”, “SLPL”, “SLPF” and “SELP” (from Protein Polymer Technologies, Inc. San Diego, Calif.). ELP's, SLP's, CLP's, SLPL's, SLPF's and SELP's are families of genetically engineered protein polymers consisting of silk-like blocks, elastin-like blocks, collagen-like blocks, laminin-like blocks, fibronectin-like blocks and the combination of silk-like and elastin-like blocks, respectively. The ELP's, SLP's, CLP's, SLPL's, SLPF's and SELP's are produced in various block lengths and compositional ratios. Generally, blocks include groups of repeating amino acids making up a peptide sequence that occurs in a protein.

The force binding the drug to the particle or the drug to the particle coating may be achieved through intra- and inter-molecular forces (ie., ionic, dipole-dipole, such as hydrogen bonding, London dispersion, hydrophobic, etc.).

One of the problems associated with the use of drug delivery implants is the exposure of the patient to risk of infection and other medical problems, such as pain and inflammation. To overcome this problem, in one embodiment of the invention a device comprising a combination of depolymerized chitosan and a drug, which may be ionically bonded to each other, is utilized.

Additionally, hydrophobic substances, such as lipids, may be incorporated into the biodegrable matrix of the devices of the present invention to extend the duration of drug release, while hydrophilic, polar additives, such as salts and amino acids, may be added to facilitate, i.e., shorten the duration of, drug release. Exemplary hydrophobic substances for use herein include lipids, e.g., tristeafin, ethyl stearate, phosphotidycholine, polyethylene glycol (PEG); fatty acids, e.g., sebacic acid erucic acid; and combinations of these and the like.

The controlled release of a drug in a drug delivery device is partially attributed to the homogenous distribution of the pharmacologically active agent(s) throughout the drug delivery device. This homogenous distribution provides for a more systematic, sustainable and consistent release of the pharmacologically active agent(s) by gradual degradation of the device matrix or diffusion of the pharmacologically active agent(s) out of the device. As a result, the release characteristics of the pharmacologically active agent from the device material and/or device are enhanced.

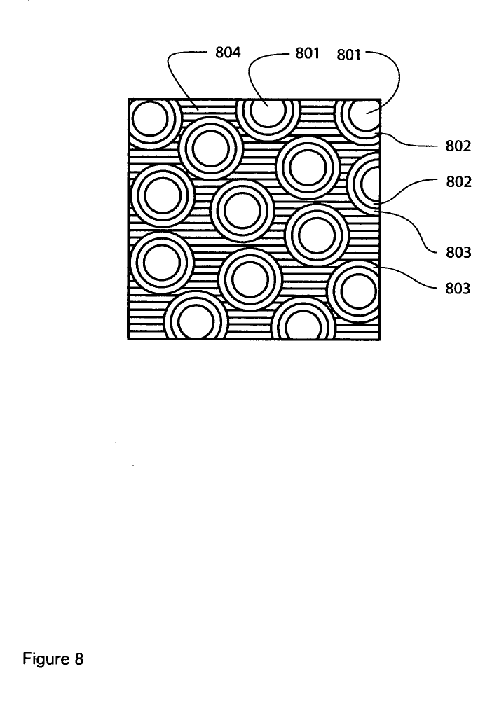

FIG. 8 illustrates a material matrix 800 of a device that comprises a coated particle 801. In this particular embodiment, the particles are perfectly spherical shaped and all have the same diameter, which may be different for other material matrices. Particle 801 is coated with a binding material 802, which binds the particle 801 to a drug 803, which is coated onto the binding layer 802. The coated particle is incorporated into a biodegradable matrix 804. In this particular case, the matrix 804 comprises elastin and hydroxapatite, which resorb in 30 days. The resorbtion rate of this composition over time depends on the inter-particle average distance, which determines how quickly the body fluids can reach the matrix composition to absorb it. In one embodiment, the drug layer is Taxol®. Some of the particles may have a third layer on top of the drug layer 803, wherein this third layer comprises a slow resorbing material to extend the time of drug elution of the device. In one embodiment, the binding layer 802 is dextran. Typically, the thickness of any of the layers ranges from about 5 nanometers to about 100 nanometers, preferably from about 20 nanometers to about 30 nanometers. In one embodiment, the particles 801 comprise iron-oxide and have a diameter of about 500 nanometers. The size is selected to ensure that particles 801 can pass through the extra-cellular space when they loosen and dissolve from the device, and are removed via digestion in the body's metabolism.

Each of the RLS, FLS and PLS configured devices may also comprise a drug releasing substance, which along with the drug, dissolves and vanishes from the body over a period of time.

4. Matrix Material

The implantable devices of the present invention comprise a matrix material, preferably a biodegradable material. The matrix material may be a polymeric material, a metallic material, or a combination of polymeric and metallic materials.

A. Biodegradable Polymer Matrix

There are various biodegradable materials on the market. Materials suitable for use as a matrix for the drug delivery devices of the present invention include, but are not limited to, the copolymers polylactides poly(L-lactide) or poly(D-lactidepoly) or copolymers derived therefrom, such as poly(L-lactide-co-D,L-lactide), poly(L-lactide-co-meso-lactide), poly(L-lactide-co-glycolide), poly(L-lactide-co-trimethylene carbonate), poly(L-lactide-co-ε.-caprolactone), poly(D,L-lactide-co-meso-lactide), poly(D,L-lactide-co-glycolide), poly(D,L-lactide-co-trimethylene carbonate), poly(D,L-lactide-co-ε-caprolactone), poly(meso-lactide-co-glycolide), poly(meso-lactide-co-trimethylene carbonate), poly(meso-lactide-co ε-caprolactone), poly(glycolide-co-trimethylene carbonate), poly(glycolide-co ε-caprolactone), and mixtures thereof. These materials may be purchased as resomers (for example from Boehringer in Ingelheim, Germany).

Other examples of biodegradable and/or biocompatible polymeric materials suitable for use herein include, but are not limited to, epoxies, polyesters, acrylics, nylons, silicones, polyanhydride, polyurethane, polycarbonate, poly(tetrafluoroethylene) (PTFE), polyethylene oxide, polycaprolactone, polyethylene glycol, poly(vinyl chloride), polylactic acid, polyglycolic acid, polypropylene oxide, sebacic acid, poly(akylene)glycol, polyoxyethylene, polyvinyl alcohol (PVA), polymethyl methacrylate, 2-hydroxyethyl methacrylate (HEMA), 1,3-bis(carboxyphenoxy)propane, lipids, poly(ethylene oxide) (PEO), polyhydroxybutyrate (PHB), phosphatidylcholine, triglycerides, poly ortho esters, polyhydroxyvalerate (PHV), poly(amino acids), polycynoacrylates, polyphophazenes, polysulfone, polyamine, poly (amido amines), fibrin, graphite, flexible fluoropolymer, isobutyl-based, isopropyl styrene, vinyl pyrrolidone, cellulose acetate dibutyrate, silicone rubber, copolymers thereof, and the like.

The biodegradable polymeric matrix material may be a poly(α-hydroxy acid) or a poly(ethylene carbonate). In one embodiment of the present invention, a drug, which is incorporated into the matrix material, is released at a controlled rate from the copolymer, which biodegrades into non-toxic products. The degradation rate may be adjusted by proper selection of the polymeric material.

In one embodiment of the present invention, the device is constructed from an elastomeric material, wherein the elastomeric material is a siloxane-based elastomer comprising 3,3,3-trifluoropropyl groups attached to the Si-atoms of the siloxane units, and wherein the elastomer comprises either (i) a mixture comprising a) a non-fluorosubstituted siloxane-based polymer and b) a fluorosubstituted siloxane-based polymer, said polymer comprising 3,3,3-trifluoropropyl groups attached to the Si-atoms of the siloxane units; or (ii) a single siloxane-based polymer comprising 3,3,3-trifluoropropyl groups attached to the Si-atoms of the siloxane units, wherein said polymer or mixture of polymers are crosslinked to form the elastomer.

The biodegradable matrix may comprise one or more biodegradable microparticles that provide greater strength to the device. The microparticles may comprise a metal, a plastic, a ceramic, or a combination thereof. These microparticles are so small that they are removed from the body in a natural way, in which the body's metabolism detects and removes unfamiliar or exotic substances. The mixture may be clustered together with an oil-in-water emulsion.

In one embodiment of the present invention, the RLS or the holding structure of the FLS may comprise biodegradable, elastic shape-memory materials. The transition from the temporary to the permanent shape of a thermally induced shape-memory material is initiated by an external stimulus, such as a temperature increase above the switching transition temperature Tₜᵣₐₙₛ of the material. All of these materials are non-degradable in physiological environments and many lack biocompatibility or compliance in mechanical properties. Polymeric materials that are designed to exhibit a thermally induced shape-memory effect require two components on the molecular level: cross-links to determine the permanent shape and switching segments with Tₜᵣₐₙₛ to fix the temporary shape. Above Tₜᵣₐₙₛ, the permanent shape may be deformed by application of an external stress. After cooling below Tₜᵣₐₙₛ and the subsequent release of the external stress, the temporary shape is obtained. Hence, instead of using heat to harden a matrix material of the RLS or FLS, a shape-memory effect approach may be utilized.

In an embodiment of the present invention, the polymeric matrix may comprise a copolymer of (a) a (meth)acrylate copolymer containing ammonio groups, or (b) a mixture of a (meth)acrylate copolymer containing amino groups and a (meth)acrylate polymer containing carboxyl groups.

In another embodiment of the invention, the polymeric matrix may comprise a polyurethane elastomeric composition that comprises a soft segment derived from at least one polysiloxane macrodiol and at least one polyether and/or polycarbonate macrodiol. The polyurethane elastomeric composition comprises a soft segment derived from about 60 wt % to about 98 wt % of at least one polysiloxane macrodiol and about 2 wt % to about 40 wt % of at least one polyether and/or polycarbonate macrodiol.

The bioresorbable polymeric material for use herein may be hydroxapatite.

B. Biodegradable Metal Matrix

It is known that certain metal alloys are biocompatible and bioresorbable. Such alloys comprise manganese in which lithium is incorporated at about 0.5 wt % to about 20 wt %.

Other metallic materials suitable for use herein include any other biocompatible and biodegradable alloy.

The matrix material of the devices disclosed herein may comprise metallic alloys that exhibit shape-memory effect. The shape-memory effect is due to a martensitic phase transition.

5. Drugs

The devices of the present invention comprise one or more drugs for the treatment or prevention of cardiovascular or vascular diseases, such as calcified or vulnerable plaque, and arteriosclerosis. As there are many diseases that are related to inflammation, these devices may also comprise one or more drugs for treating or preventing inflammation to treat or prevent vascular or cardiovascular diseases, rheumatoid arthritis, diabetes, or Alzheimer disease.

In one embodiment of the present invention, the drug is Taxol® or Paclitaxel™. The drug may be mixed into the matrix material on a molecular or small droplet basis. The size of each droplet ranges from about 10 micrometers to about 100 micrometers. These droplets work as little drug depots and open to release the drug when the material of the matrix vanishes over time. In one embodiment, Zyn-Linkers are used to modify the delivery of the drug. Zyn-Linkers are small molecules, which, when chemically coupled to therapeutic agents, anchor them at target sites in the body and release the therapeutic agents at controlled rates over long periods, and thereby reducing the number of required doses and decreasing the side effects.

Other drugs useful herein, include dexamethasone, rapamicine, tacrolimus, polymer-based copper nitric oxide from S-nitrosoglutathione, and 17-beta-estradiol.

Drugs useful herein for preventing or treating inflammation include, but are not limited to, Avastin™ (from Genentech), Velvode (from Millenium Pharmaceuticals), aspirin, statins, beta blockers, and ACE inhibitors.

Other drugs suitable for use herein are listed in Table I.

TABLE I
DRUGS SUITABLE FOR USE IN THE PRESENT INVENTION

| Drug | Description |
|---|---|
| Adenosine (ATP) | Antiarrhythmic, first line drug used for termination of Supraventricular |
| | Tachycardias (SVT) involving the AV node or the accessory pathways |
| | (WPW). It can also block the AV node transiently to facilitate the |
| | interpretation of the surface ECG. |
| Aldactone ® | A diuretic to treat heart failure and fluid retention due to cirrhosis of liver. |
| | Recent study (RALES) showed that it is useful for heart failure patients. |
| Alteplace tPA (t-PA) | Thrombolytic. Used for lysis of clot inside the coronary vessels in acute |
| | myocardial infarction; it can also be used for treating pulmonary embolism |
| Amlodipine | Calcium Channel Blocker (CCB), 2nd generation. Used for treatment of |
| | hypertension, ischemic heart disease and angina. |
| Amiodarone | Class III anti-arrhythmic. Used for terminating and preventing |
| | supraventricular arrhythmias (SVT) including atrail fibrillation and |
| | ventricular arrhythmias (VT) |
| Anistreplase (APSAC) | Thrombolytic for lysis of clot in the coronary vessels in acute myocardial |
| | infarction. |
| Aspirin | Analgesic. Used also for reducing risk of myocardial infarction and risk of |
| | death after infarction or angina. Also used for reducing risk of |
| | thromboembolism in high risk patients. |
| Atenolol | Beta blocker. Used for treaof hypertension, ischemic heart disease, |
| | angina, post myocardial infarction, and heart failure. |
| Atropine | Anticholinergic. used for treatment of bradycardia and heart blocks |
| Abciximab (Reopro) | A new Glycoprotein IIb/IIIa receptor antagonist. Used for complicated |
| | PTCA/PTCS procedures, also studied for use in unstable angina and acute |
| | myocardial infarction. |
| Captopril | Angiotensin Coverting Enzyme Inhibitor (ACEI). Used for treatment of |
| | hypertenison, heart failure and post myocardial infarction remodelling. |
| Carvedilol | Alpha & Beta-blocker with vasodilor activity. Used for treatment of |
| | congestive heart failure. Start at low dose and titrate up slowly. New studies |
| | show that it reduces mortality in Class II-IV heart failure patients. |
| Celebrex ® | Used to treat inflammation. |
| Chlorothiazide | Used for treatment of hypertension and heart failure. |
| Cholestyramine | Bile acid sequestrant. Used for treatment of hyperlipidaemia. |
| Clofibrate | Fibric acid derivative. Used for treatment of hyperlipideamia. |
| Clopidrogel | A new anti-platelet (acts on ADP receptor) with action similar to |
| | ticlodipine. Used for angina, PTCA/S procedures and strokes. New studies |
| | show that it may be useful for unstable angina and myocardial infarction. |
| Digoxin | Digitalis. Used for the control of ventricular rate in atrial fibrillation, heart |
| | failure and PAF. |
| Dipyridamole | Antiplatelet. Used for prevention of thromboembolic disease, cardiac |
| | valvulaer replacement, and stenting. |
| Disopyramide | Class Ia anti-arrhythmic. Used for treatment of atrial and ventricular |
| | arrhythmias. |
| Dobutamine | Inotopic agent. Used for blood pressure support, and hypotension. |
| Dofetilide | Used for treatment of AF and restoration of normal cardiac rhythm. |
| Dopamine | Inotopic agent. Used for blood pressure support, hypotension, and renal |
| | vascular perfusion (low dose). |
| Enalapril | Angiotensin Coverting Enzyme Inhibitor. Used for treatment of |
| | hypertenison, heart failure and post myocardial infarction remodelling. |
| Epinephrine | Vasopressor. Used for treatment of hypotension and shock, ventricular |
| | fibrillation, asystole, cardiac arrest, bradycardia, analyphylactic shock |
| Felodipine | Calcium Channel blocker (CCB). Used for treatment of hypertension, |
| | ischemic heart disease and angina. |
| Flecainide | Class Ic antiarrhythmic. Used for treatment of atrial and ventricular |
| | arrhythmias. |
| Furosemide | Loop diuretics. Used for treatment of hypertension and heart failure. |
| Heparin | Anticoagulant. Used for treatment of deep vein thrombisis, pulmonary |
| | embolism, acute myocardial infarction, unstable angina, and peripheral |
| | vessel embolism. |
| Heparin | Anticoagulant. Prophylaxis of deep vein thrombosis and pulmonary |
| | embolism. Also used after PTCA/S. |
| Hydralazine | Direct vasodilator. Used for treatment of malignant hypertension, heart |
| | failure, pre-eclampsia, and eclampsia. |
| Ibutilide | Class III antiarrhythmic. Preparation for acute conversion of atrial |
| | fibrillation or flutter. |
| Isosorbide dinitrate | Nitrate. Used for treatment of angina and ischemic heart disease. |
| Labetalol | Alpha and beta blocker. Used for treatment of hypertension, |
| | pheochromocytoma and dissecting aortic aneurysm. |
| Lidocaine | Class Ib anti-arrhythmic. Treatment of ventricular arrhythmiac, ventricular |
| | fibrillation. |
| Lisinopril | Angiotensin Coverting Enzyme Inhibitor. Used for treatment of |
| | hypertenison, heart failure and post myocardial infarction remodelling |
| Losartan | Ang II receptor antagonist. Used for treatment of hypertension, may also be |
| | used for heart failure |
| Lovastatin | HMGCoA reductase inhibitor. Used for treatment of hyperlipidemia. |
| Methydopa | Alpha-blocker (central). Used for treatment of hypertension. |
| Metoprolol | Beta-1-selective blocker. Used for treatment of hypertension, ischemic |
| | heart disease and post myocardial infarction decrease in mortality. |
| Minoxidil | Direct vasodilator. Used for treatment of hypertension and heart failure. |
| Nifedipine | Calcium Channel Blocker. Used for treatment of hypertension, ischemic |
| | heart disease and angina. |
| Nimodipine | Calcium Channel Blocker. Used for treatment of hypertension, ischemic |
| | heart disease and angina. |
| Nitropusside | Direct vasodilator. Used for treatment of hypertension, heart failure and |
| | dissecting aorta aneurysm. |
| Pravastatin | HMGCoA reductase inhibitor. Used for treatment of hyperlipidemia. |
| Procainamide | Class Ia antiarrhythmoc. Used for treatment of atrail and ventricular |
| | arrhythmias. |
| Propranolol | Beta-blocker. Used for treatment of hypertension, ischemic heart disease, |
| | angina, post myocardial infarction, and heart failure. |
| Protamine | Heparin antagonist. Reversal of heparin anticoagulation and treatment of |
| | overdose. |
| Simvastatin | HMGCoA reductase inhibitor. Used for treatment of hyperlipidemia. |
| Sotalol | Class II and III anti-arrhythmic. Used for treatment of supraventricular |
| | arrhythmia and ventricular arrhythmia. |
| Streptokinase | Thrombolytic. Used for treatment of acute myocardial infarction (onset of |
| | chest pain less than 12 hours) and pulmonary embolism. |
| Ticlodipine | Antiplatelet agent. Used for stroke prevention and thromboembolic disease, |
| | also used for PTCA and stenting procedure. |
| Urokinase | Thrombolytic. Used for treatment of acute myocardial infarction (onset of |
| | chest pain less than 12 hours) and pulmonary embolism. |
| Verapamil | Calcium Channel Blocker. Used for treatment of hypertension, angina and |
| | atrial arrhythmias. |
| Warfarin | Anticoagulant. Used for prophylaxis and treatment of thromboembolic |
| | disease, and pulmonary embolism. |

Additional drugs suitable for use herein can be found in “Today in Cardiology”, January 2003 edition, pages 15 to 17, [published by SLACK Inc., 6900 Grove Road, Thorofare, N.J. 08086 USA], which are incorporated herein by reference.

Lactate metal salts, aminoguanidinyl- and alkoxyguanidinyl-substituted phenyl acetamides, 7-oxo-pyridopyrimidines (II), and squaric acid derivatives may also be suitable for use herein. Lactate metal salt, in particular an L-lactate, may also be used for the treatment of arteriosclerosis and/or for the prophylaxis or treatment of diseases caused by arteriosclerosis. Aminoguanidinyl- and alkoxyguanidinyl-substituted phenyl acetamides may be used as protease inhibitors. 7-oxo-pyridopyrimidines (II) may be used as an anti-inflammatory drug. Squaric acid derivatives are able to inhibit the binding of integrins to their ligands and thus are useful in the prophylaxis and treatment of immune of inflammatory disorders, or disorders involving the inappropriate growth or migration of cells.

By reducing LDL cholesterol or other lipids, plaque build-up may be prevented or even reduced. And within a few months of treatment, plaques may be stabilized. Numerous studies have demonstrated that lowering cholesterol can reduce the risk of heart attack and death in people at high risk of a heart attack. The following types of drugs, resins, fibrates, niacin or statins, are useful herein for lowering cholesterol.

Resins: Cholestyramine (Questran®) and colestipol (Colestid®), both lower cholesterol indirectly by binding with bile acids in the intestinal tract. Bile acids are produced in the liver from cholesterol and are needed for food digestion. By tying up bile acids, the drugs prompt the liver to produce more bile acids. Because the liver uses cholesterol to make the acids, less cholesterol is available to reach the bloodstream.

Fibrates: Gemfibrozil (Lopid®), fenofibrate (Tricor®), and bezafibrate are triglyceride-lowering drugs that also increase the levels of good cholesterol (HDL). They are also referred to as fibric acid derivatives. They reduce triglyceride production and remove triglycerides from circulation.

Niacin: Large doses of niacin, a vitamin, also can lower triglycerides. In addition, niacin can lower LDL cholesterol and increase HDL cholesterol; both have beneficial effects.

Statins: These drugs, introduced in the late 1980s, are fast becoming the most widely prescribed drugs to lower cholesterol. They are also referred to as HMG-CoA reductase inhibitors. Examples are: fluvastatin (Lescol®), lovastatin (Mevacor®), simvastatin (Zocor®), pravastatin (Pravachol®), atorvastatin (Lipitor®), and cerivastatin. Statins work directly in the liver to block a substance the liver needs to manufacture cholesterol which depletes cholesterol in the liver cells and causes the cells to remove cholesterol from circulating blood. Depending on the dose, statins can reduce LDL cholesterol by up to 40 percent. Statins may also help the body to reabsorb cholesterol from plaques, which slowly unplugs the blood vessels. Statins reduce inflammation around the plaques, which helps to stabilize them and reduces the chances of rupture and blockage of the affected artery. Statins are the only type of lipid-lowering drug proven to reduce the risk of death from cardiovascular disease. Along with niacin, statins have also been proven to reduce the risk of having a second heart attack.

Another drug useful herein is colesefibrozil.

Meso-formyl porphyrins, meso-acrylate porphyrins, purpurins and benzochlorins and mono-formylated tetrapyrrolic may have a healing effect on calcified and vulnerable plaque. In one embodiment of the present invention, meso-formyl porphyrins, meso-acrylate porphyrins, purpurins, benzochlorins, mono-formylated tetrapyrrolic, or a combination thereof is used as the drug in the devices of the present invention.

In another embodiment, the drug used is tamoxifen, which is widely used for breast cancer.

Other pharmacologically active agents suitable for use herein are as follows:

Antidiarrhoeals, such as diphenoxylate, loperamide and hyoscyamine;

Antihypertensives, such as hydralazine, minoxidil, captopril, enalapril, clonidine, prazosin, debrisoquine, diazoxide, guanethidine, methyldopa, reserpine, and trimethaphan;

Calcium channel blockers, such as diltiazem, felodipine, amlodipine, nitrendipine, nifedipine and verapamil;

Antiarrhyrthmics, such as amiodarone, flecainide, disopyramide, procainamide, mexiletene and quinidine;

Antiangina agents, such as glyceryl trinitrate, erythrityl tetranitrate, pentaerythritol tetranitrate, mannitol hexanitrate, perhexilene, isosorbide dinitrate and nicorandil;

Beta-adrenergic blocking agents, such as alprenolol, atenolol, bupranolol, carteolol, labetalol, metoprolol, nadolol, nadoxolol, oxprenolol, pindolol, propranolol, sotalol, timolol and timolol maleate;

Cardiotonic glycosides, such as digoxin and other cardiac glycosides and theophyllne derivatives;

Adrenergic stimulants, such as adrenaline, ephedrine, fenoterol, isoprenaline, orciprenaline, rimeterol, salbutamol, salmeterol, terbutaline, dobutamine, phenylephrine, phenylpropanolamine, pseudoephedrine and dopamine;

Vasodilators, such as cyclandelate, isoxsuprine, papaverine, dipyrimadole, isosorbide dinitrate, phentolamine, nicotinyl alcohol, co-dergocrine, nicotinic acid, glycerl trinitrate, pentaerythritol tetranitrate and xanthinol;

Antimigraine preparations, such as ergotanmine, dihydroergotamine, methysergide, pizotifen and sumatriptan;

Anticoagulants and thrombolytic agents, such as warfarin, dicoumarol, low molecular weight hepafins such as enoxaparin, streptokinase and its active derivatives;

Hemostatic agents, such as aprotinin, tranexamic acid and protarnine;

Analgesics and antipyretics including the opioid analgesics, such as buprenorphine, dextromoramide, dextropropoxyphene, fentanyl, alfentanil, sufentanil, hydromorphone, methadone, morphine, oxycodone, papaveretum, pentazocine, pethidine, phenopefidine, codeine dihydrocodeine; acetylsalicylic acid (aspirin), paracetamol, and phenazone;

Neurotoxins, such as capsaicin;

Hypnotics and sedatives, such as the barbiturates amylobarbitone, butobarbitone and pentobarbitone and other hypnotics and sedatives such as chloral hydrate, chlormethiazole, hydroxyzine and meprobamate;

Antianxiety agents, such as the benzodiazepines alprazolam, bromazepam, chlordiazepoxide, clobazam, chlorazepate, diazepam, flunitrazepam, flurazepam, lorazepam, nitrazepam, oxazepam, temazepam and triazolam;

Neuroleptic and antipsychotic drugs, such as the phenothiazines, chlorpromazine, flupbenazine, pericyazine, perphenazine, promazine, thiopropazate, thioridazine, trifluoperazine; and butyrophenone, droperidol and haloperidol; and other antipsychotic drugs, such as pimozide, thiothixene and lithium;

Antidepressants, such as the tricyclic antidepressants amitryptyline, clomipramine, desipramine, dothiepin, doxepin, imipramine, nortriptyline, opipramol, protriptyline and trimipramine and the tetracyclic antidepressants such as mianserin and the monoamine oxidase inhibitors such as isocarboxazid, phenelizine, tranylcypromine and moclobemide and selective serotonin re-uptake inhibitors, such as fluoxetine, paroxetine, citalopram, fluvoxamine and sertraline;

CNS stimulants, such as caffeine and 3-(2-aminobutyl)indole;

Anti-alzheimer's agents, such as tacrine;

Anti-Parkinsons agents, such as amantadine, benserazide, carbidopa, levodopa, benztropine, bipefiden, benzhexol, procyclidine and dopamine-2 agonists-;

Anticonvulsants, such as phenytoin, valproic acid, primidone, phenobarbitone, methylphenobarbitone and carbamazepine, ethosuximide, methsuximide, phensuximide, sulthiame and clonazepam,

Antiemetics and antinauseants, such as the phenothiazines prochloperazine, thiethylperazine and 5HT-3 receptor antagonists, such as ondansetron and granisetron, as well as dimenhydrinate, diphenhydramine, metoclopramide, domperidone, hyoscine, hyoscine hydrobromide, hyoscine hydrochloride, clebopride and brompride;

Non-steroidal anti-inflammatory agents including their racemic mixtures or individual enantiomers where applicable, preferably formulated in combination with dermal penetration enhancers, such as ibuprofen, flurbiprofen, ketoprofen, aclofenac, diclofenac, aloxiprin, aproxen, aspirin, diflunisal, fenoprofen, indomethacin, mefenamic acid, naproxen, phenylbutazone, piroxicam, salicylamide, salicylic acid, sulindac, desoxysulindac, tenoxicam, tramadol, ketoralac, flufenisal, salsalate, triethanolamine salicylate, atninopyrine, antipyrine, oxyphenbutazone, apazone, cintazone, flufenamic acid, clonixerl, clonixin, meclofenamic acid, flunixin, colchicine, demecolcine, allopurinol, oxypurinol, benzydamine hydrochloride, dimefadane, indoxole, intrazole, mimbane hydrochloride, paranylene hydrochloride, tetrydamine, benzindopyrine hydrochloride, fluprofen, ibufenac, naproxol, fenbufen, cinchophen, diflumidone sodium, fenamole, flutiazin, metazamide, letimide hydrochloride, nexeridine hydrochloride, octazamide, molinazole, neocinchophen, nimazole, proxazole citrate, tesicam, tesimide, tolmetin, and triflumidate;

Antirheumatoid agents, such as penicillamine, aurothioglucose, sodium aurothiomalate, methotrexate and auranofin;

Muscle relaxants, such as baclofen, diazepam, cyclobenzaprine hydrochloride, dantrolene, methocarbamol, orphenadrine and quinine;

Agents used in gout and hyperuricaemia, such as allopurinol, colchicine, probenecid and sulphinpyrazone;

Oestrogens, such as oestradiol, oestriol, oestrone, ethinyloestradiol, mestranol, stilboestrol, dienoestrol, epioestriol, estropipate and zeranol;

Progesterone and other progestagens, such as allyloestrenol, dydrgesterone, lynoestrenol, norgestrel, norethyndrel, norethisterone, norethisterone acetate, gestodene, levonorgestrel, medroxyprogesterone and megestrol;

Antiandrogens, such as cyproterone acetate and danazol;

Antioestrogens, such as tamoxifen and epitiostanol and the aromatase inhibitors, exemestane and 4-hydroxy-androstenedione and its derivatives;

Androgens and anabolic agents, such as testosterone, methyltestosterone, clostebol acetate, drostanolone, furazabol, nandrolone oxandrolone, stanozolol, trenbolone acetate, dihydro-testostero 17-(a-methyl-19-noriestosterone and fluoxymesterone;

5-alpha reductase inhibitors, such as finastride, turosteride, LY-191704 and MK-306-1;

Corticosteroids, such as betamethasone, betamethasone valerate, cortisone, dexamethasone, dexamethasone 21-phosphate, fludrocortisone, flumethasone, fluocinonide, fluocinonide desonide, fluocinolone, fluocinolone acetonide, fluocortolone, halcinonide, halopredone, hydrocortisone, hydrocortisone 17-valerate, hydrocortisone 17-butyrate, hydrocortisone 21-acetate, methylprednisolone, prednisolone, prednisolone 21-phosphate, prednisone, triamcinolone, triamcinolone acetonide;

Glycosylated proteins, proteoglycans, glycosaminoglycans such as chondroitin sulfate; chitin, acetyl-glucosamine, and hyaluronic acid;

Complex carbohydrates, such as glucans;

Anti-inflammatory drugs (anti-inflammatory drugs to prevent or treat vascular plaque, such as calcified or vulnerable plaque, or Alzheimer's disease), such as Celebrex® from Pfizer;

Steroidal anti-inflammatory agents, such as cortodoxone, fludroracetonide, fludrocortisone, difluorsone diacetate, flurandrenolone acetonide, medrysone, amcinafel, amcinafide, betamethasone and its other esters, chloroprednisone, clorcortelone, descinolone, desonide, dichlofisone, difluprednate, flucloronide, flumethasone, flunisolide, flucortolone, fluoromethalone, fluperolone, fluprednisolone, meprednisone, methylmeprednisolone, paramethasone, cortisone acetate, hydrocortisone cyclopentylpropionate, cortodoxone, flucetonide, fludrocortisone acetate, amcinafal, amcinafide, betamethasone, betamethasone benzoate, chloroprednisone acetate, clocortolone acetate, descinolone acetonide, desoximetasone, dichlorisone acetate, difluprednate, flucloronide, flumethasone pivalate, flunisolide acetate, fluperolone acetate, fluprednisolone valerate, paramethasone acetate, prednisolamate, prednival, triamcinolone hexacetonide, cortivazol, formocortal and nivazoll;

Pituitary hormones and their active derivatives or analogs, such as corticotrophin, thyrotropin, follicle stimulating hormone (FSH), luteinising hormone (LH) and gonadotrophin releasing hormone (GnRH);

Hypoglycemic agents, such as insulin, chlorpropamide, glibenclamide, gliclazide, glipizide, tolazamide, tolbutamide and metformin;

Thyroid hormones, such as calcitonin, thyroxine and liothyronine and antithyroid agents such as carbimazole and propylthiouracil;

Other miscellaneous hormone agents, such as octreotide;

Pituitary inhibitors, such as bromocriptine;

Ovulation inducers, such as clomiphene;

Diuretics, such as the thiazides, related diuretics and loop diuretics, bendrofluazide, chlorothiazide, chlorthalidone, dopamine, cyclopenthiazide, hydrochlorothiazide, indapamide, mefruside, methycholthiazide, metolazone, quinethazone, bumetanide, ethacrynic acid and frusemide and potasium sparing diuretics, spironolactone, amiloride and triamterene;

Antidiuretics, such as desmopressin, lypressin and vasopressin including their active derivatives or analogs;

Obstetric drugs including agents acting on the uterus, such as ergometfine, oxytocin and gemeprost;

Prostaglandins, such as alprostadil (PGEI), prostacyclin (PG12), dinoprost (prostaglandin F2-alpha) and misoprostol;

Antimicrobials, including the cephalospofins such as cephalexin, cefoxytin and cephalothin;

Penicillins, such as amoxycillin, amoxycillin with clavulanic acid, ampicillin, bacampicillin, benzathine penicillin, benzylpenicillin, carbenicillin, cloxacillin, methicillin, phenethicillin, phenoxymethylpenicillin, flucloxacillin, meziocillin, piperacillin, ticarcillin and azlocillin;

Tetracyclines, such as minocycline, chlortetracycline, tetracycline, demeclocycline, doxycycline, methacycline and oxytetracycline and other tetracycline-type antibiotics;

Amnioglycoides, such as amikacin, gentamicin, kanamycin, neomycin, netilmicin and tobramycin;

Antifingals, such as amorolfine, isoconazole, clotrimazole, econazole, miconazole, nystatin, terbinafine, bifonazole, amphotericin, griseofulvin, ketoconazole, fluconazole and flucytosine, salicylic acid, fezatione, ticlatone, toinaftate, triacetin, zinc, pyrithione and sodium pyfithione;

Quinolones, such as nalidixic acid, cinoxacin, ciprofloxacin, enoxacin and norfloxacin;

Sulphonamides, such as phthalysulphthiazole, sulfadoxine, sulphadiazine, sulphamethizole and sulphamethoxazole;

Sulphones, such as dapsone;

Antibiotics, such as chloramphenicol, clindamycin, erythromycin, erythromycin ethyl carbonate, erythromycin estolate, erythromycin glucepate, erythromycin ethylsuccinate, erythromycin lactobionate, roxithromycin, lincomycin, natamycin, nitrofurantoin, spectinomycin, vancomycin, aztreonarn, colistin IV, metronidazole, tinidazole, fusidic acid, trimethoprirn, and 2-thiopyridine N-oxide; halogen compounds, particularly iodine and iodine compounds such as iodine-PVP complex and diiodohydroxyquin, hexachlorophene; chlorhexidine; chloroan-tine compounds; and benzoylperoxide;

Antituberculosis drugs, such as ethambutol, isoniazid, pyrazinamide, rifampicin and clofazimine;

Antimalarials, such as primaquine, pyrimethamine, chloroquine, hydroxychloroquine, quinine, mefloquine and halofantrine;

Antiviral agents, such as acyclovir and acyclovir prodrugs, famcyclovir, zidovudine, didanosine, stavudine, lamivudine, zalcitabine, saquinavir, indinavir, ritonavir, n-docosanol, tromantadine and idoxuridine;

Anthelmintics, such as mebendazole, thiabendazole, niclosamide, praziquantel, pyrantel embonate and diethylcarbamazine;

Cytotoxic agents, such as plicamycin, cyclophosphamide, dacarbazine, fluorouracil and its prodrugs, methotrexate, procarbazine, 6-mercaptopurine and mucophenolic acid;

Anorectic and weight reducing agents, including dexfenflurarnine, fenfluramine, diethylpropion, mazindol and phentermine;

Agents used in hypercalcaemia, such as calcitriol, dihydrotachysterol and their active derivatives or analogs;

Antitussives, such as ethylmorphine, dextromethorphan and pholcodine;

Expectorants, such as carbolcysteine, bromhexine, emetine, quanifesin, ipecacuanha and saponins;

Decongestants, such as phenylephrine, phenylpropanolamine and pseudoephedrine;

Bronchospasm relaxants, such as ephedrine, fenoterol, orciprenaline, rimiterol, salbutamol, sodium cromoglycate, cromoglycic acid and its prodrugs, terbutailne, ipratropium bromide, salmeterol and theophylline and theophylline derivatives;

Antihistamines, such as meclozine, cyclizine, chlorcyclizine, hydroxyzine, brompheniramine, chlorpheniramiine, clemastine, cyproheptadine, dexchlorpheniramine, diphenhydramine, diphenylamine, doxylatnine, mebhydrolin, pheniramine, tripolidine, azatadine, diphenylpyraline, methdilazine, terfenadine, astemizole, loratidine and cetirizine;

Local anaesthetics, such as bupivacaine, amethocaine, lignocaine, lidocaine, cinchocaine, dibucaine, mepivacaine, prilocaine, etidocaine, veratridine (specific c-fiber blocker) and procaine;

Stratum corneum lipids, such as ceramides, cholesterol and free fatty acids, for improved skin barrier repair;

Neuromuscular blocking agents, such as suxamethonium, alcuronium, pancuronium, atracurium, gallamine, tubocurarine and vecuronium;

Smoking cessation agents, such as nicotine, bupropion and ibogaine;

Insecticides and other pesticides which are suitable for local application;

Dermatological agents, such as vitamins A, C, B1, B2, B6, B12, and E, vitamin E acetate and vitamin E sorbate;

Allergens for desensitisation, such as house, dust or mite allergens;

Nutritional agents and neutraceuticals, such as vitamins, essential amino acids and fats;

Acromolecular pharmacologically active agents, such as proteins, enzymes, peptides, polysaccharides (such as cellulose, amylose, dextran, chitin), nucleic acids, cells, tissues, and the like; and

Keratolytics, such as the alpha-hydroxy acids, glycolic acid and salicylic acid.

The devices of the present invention may comprise a pharmaceutical composition comprising acarbose; acyclovir; acetyl cysteine; acetylcholine chloride; alatrofloxacin; alendronate; alglucerase; amantadine hydrochloride; ambenomium; amifostine; amiloride hydrochloride; aminocaproic acid; amphotericin B; antihemophilic, factor (human); antihemophilic factor (porcine); antihemophilic factor (recombinant); aprotinin; asparaginase; atenolol; atracurium besylate; atropine; azithromycin; aztreonam; BCG vaccine; bacitracin; becalermin; belladona; bepridil hydrochloride; bleomycin sulfate; calcitonin human; calcitonin salmon; carboplatin; capecitabine; capreomycin sulfate; cefamandole nafate; cefazolin sodium; cefepime hydrochloride; cefixime; cefonicid sodium; cefoperazone; cefotetan disodium; cefotoxime; cefoxitin sodium; ceftizoxime; ceftriaxone; cefuroxime axetil; cephalexin; cephapirin sodium; cholera vaccine; chrionic gonadotropin; cidofovir; cisplatin; cladribine; clidinium bromide; clindamycin and clindamycin derivatives; ciprofloxacin; clondronate; colistimethate sodium; colistin sulfate; cortocotropin; cosyntropin; cromalyn sodium; cytarabine; daltaperin sodium; danaproid; deforoxamine; denileukin diftitox; desmopressin; diatrizoate megluamine and diatrizoate sodium; dicyclomine; didanosine; dirithromycin; dopamine hydrochloride; dornase alpha; doxacurium chloride; doxorubicin; editronate disodium; elanaprilat; enkephalin; enoxacin; enoxaprin sodium; ephedrine; epinephrine; epoetin alpha; erythromycin; esmol hydrochloride; factor IX; famiciclovir; fludarabine; fluoxetine; foscarnet sodium; ganciclovir; granulocyte colony stimulating factor; granulocyte-macrophage stimulating factor; growth hormones-recombinant human; growth hormone-bovine; gentamycin; glucagon; glycopyrolate; gonadotropin releasing hormone and synthetic analogs thereof; GnRH; gonadorelin; grepafloxacin; hemophilus B conjugate vaccine; Hepatitis A virus vaccine inactivated; Hepatitis B virus vaccine inactivated; heparin sodium; indinavir sulfate; influenza virus vaccine; interleukin-2; interleukin-3; insulin-human; insulin lispro; insulin procine; insulin NPH; insulin aspart; insulin glargine; insulin detemir; interferon alpha; interferon beta; ipratropium bromide; isofosfamide; japanese encephalitis virus vaccine; lamivudine; leucovorin calcium; leuprolide acetate; levofloxacin; lincomycin and lincomycin derivatives; lobucavir; lomefloxacin; loracarbef; mannitol; measles virus vaccine; meningococcal vaccine; menotropins; mephenzolate bromide; mesalmine; methanamine; methotrexate; methscopolamine; metformin hydrochloride; metroprolol; mezocillin sodium; mivacurium chloride; mumps viral vaccine; nedocromil sodium; neostigmine bromide; neostigmine methyl sulfate; neutontin; norfioxacin; octreotide acetate; ofloxacin; olpadronate; oxytocin; pamidronate disodium; pancuronium bromide; paroxetine; pefloxacin; pentamindine isethionate; pentostatin; pentoxifylline; periciclovir; pentagastrin; phentolamine mesylate; phenylalanine; physostigmine salicylate; plague vaccine; piperacillin sodium; platelet derived growth factor-human; pneumococcal vaccine polyvalent; poliovirus vaccine inactivated; poliovirus vaccine live (OPV); polymixin B sulfate; pralidoxine chloride; pramlintide; pregabalin; propofenone; propenthaline bromide; pyridostigmine bromide; rabies vaccine; residronate; ribavarin; rimantadine hydrochloride; rotavirus vaccine; salmetrol xinafoate; sincalide; small pox vaccine; solatol; somatostatin; sparfloxacin; spectinomycin; stavudine; streptokinase; streptozocin; suxamethonium chloride; tacrine hydrochloride; terbutaline sulfate; thiopeta; ticarcillin; tiludronate; timolol; tissue type plasminogen activator; TNFR:Fc; TNK-tPA; trandolapril; trimetrexate gluconate; trospectinomycin; trovafloxacin; tubocurarine chloride; typhoid vaccine live; urea; urokinase; vancomycin; valaciclovir; valsartan; varicella virus vaccine live; vasopressin and vasopressin derivatives; vecoronium bromide; vinblastin; vincristine; vinorelbine; vitamin B12; warfarin sodium; yellow fever vaccine; zalcitabine; zanamavir; zolandronate; zidovudine; pharmaceutically acceptable salts, isomers and derivatives thereof; or combinations thereof.

Additional pharmacologically active agents suitable for use herein include an angiogenic factor, growth factor, inotropic agent, antiatherogenic agent, anti-coagulant, anti-arrhythmic agent, sympathomimetic agent, phosphodiesterase inhibitor, antineoplastic agent, and steroids.

A drug may dilute over a time period, for example, of up to one day, one week, one month, one year, or ten years.

The devices of the present invention are useful for local delivery of drugs to treat cardiovascular or vascular diseases, such as plaques or stenosis. These devices may also be used as an alternative over stents, for patients who comprise multiple stents in the treated vessel. The devices of the present invention are also suitable for use with patients who have already undergone vascular procedures, such as a PTCA, or who are classified as high-risk patients due to their family history, their high LDL (low density lipoprotein) or CRP (C-Reactive Protein) levels.

### EXAMPLES

Having generally described the invention, a more complete understanding thereof may be obtained by reference to the following examples that are provided for purposes of illustration only and do not limit the invention.

### Example 1

### Device having an RLS Configuration

An RLS comprises an outer layer of polymeric matrix, which contains a drug of a high concentration that will elute very quickly, and an inner core of polymeric matrix contains a drug, that elutes slowly over a long period of time. This RLS is useful for treating a stenosis proximal downstream to the RLS and thus preventing the vessel part from restenosis.

### Example 2

### Device having an RLS Configuration and Comprising Two Different Drugs

An RLS comprises only one material matrix, which contains two different drugs with different wash-out-characteristics. First drug elutes very quickly, while the second drug elute slowly over time. The second drug may only elute while the matrix material of the RLS slowly elutes over time, while the first drug washed out of the matrix material quickly. This RLS is also useful for treating a stenosis proximal downstream to the RLS and thus preventing the vessel part from restenosis.

In one embodiment of the invention, the RLS comprises small depots of a liquid-like or gel-type drugs that open when the matrix material vanishes by elution.

### Example 3

### Device having an FLS Configuration

An FLS comprising a holding structure, in the shape of a ring, a metal matrix material, and fibers comprising atropine is deployed in a cardiovascular vessel. The FLS is useful for treating bradycardia and heart blocks.

### Example 4

### Device having a PLS Configuration

A PLS comprising a polymeric matrix that comprises Celebrex® is deployed, via a balloon catheter, in a renal artery. The PLS is useful for treating inflammation in the kidneys.

While the above description of the invention has been presented in terms of a human subject (patient), it is appreciated that the invention may also be applicable to treating other subjects, such as mammals, organ donors, and the like.

As noted above, the present invention is applicable to implantable devices capable of releasing a drug or pharmaceutical agent for the treatment or prevention of cardiovascular or vascular diseases, or diseases that may be attributable to inflammation, and methods related thereto. The present invention should not be considered limited to the particular embodiments described above, but rather should be understood to cover all aspects of the invention as fairly set out in the appended claims. Various modifications, equivalent processes, as well as numerous structures to which the present invention may be applicable will be readily apparent to those skilled in the art to which the present invention is directed upon review of the present specification. The claims are intended to cover such modifications and devices.

Component Numbers of Figures

| WD number | | LE number |
|---|---|---|
| 1 | ring like structure | 100 |
| 2 | balloon | 101 |
| 3 | catheter system | 102 |
| 4 | gide wire | 103 |
| 5 | syringe | 104 |
| 6 | adapter | 105 |
| 7 | vessel lumen | not used |
| 8 | vessel wall | not used, use |
| | | 106 for vessel |
| 9 | RLS in zick-zack structure | 200 |
| 10 | edges of RLS 9 | 202 |
| 11 | strap of a RLS | 301 |
| 12 | drug containing coating | 304 (drug |
| | | containing layer) |
| 13 | outer side of the strap 11 facing the vessel wall | 302 |
| | | (outer surface) |
| 14 | inner side of the strap 11 facing the blood stream | 303 |
| 15 | rough surface | 305 (inner |
| | | surface that's |
| | | been roughened) |
| 16 | coating | 306 |
| 17 | coating | 307 |
| 18 | ring | 400 (RLS) |
| 19 | operating device | not used |
| 20 | catheter | 404 |
| 21 | balloon | 402 |
| 22 | gide wire | 403 |
| 23 | vessel wall | 401 (vessel) |
| 24 | pass through hole | 405 |
| 25 | mechanical spring | 406 |
| 50 | flag like structure FLS | see below ** |
| 51 | holding structure for FLS | 502 |
| 52 | attachment of flags on holding structure | not used |
| 53 | coronary vessel | 503 |
| 54 | ascending aorta | 504 |
| 55 | arrow, pointing in the direction of the blood flow | 505 |
| 56 | flag like structure | 600 |
| 57 | sub structure of FLS 56 | 602 |
| 58 | single fibre of an FLS 57/56 | 601 |
| 59 | single fibre of an FLS 56 | 603 |
| 60 | fibre of an FLS with a taper cross-section | 604 |
| 61 | distal part of fibre 60 | 605 |
| 62 | proximal part of fibre 60 | 606 |
| 80 | plaster like structure PLS | 700 |
| 81 | balloon | 701 |
| 82 | guide wire | 703 |
| 83 | catheter system | 702 |
| 84 | syringe | 704 |
| 85 | adapter | 705 |
| 86 | blood stream | 70 |
| 87 | vessel wall | 706 (vessel) |
| 90 | particle | 801 |
| 91 | binding coating | 802 |
| 92 | drug coating | 803 |
| 93 | resorbable matrix | 804 |

NEW LE numbers to add
FIG. 2 201 for strap
FIG. 5 ** 500 is for the FLS, and 501 is for the fibers
FIG. 6 a holding structure 607, like 51 (or 502) in FIG. 5
FIG. 8 800 for PLS

1. An implantable device for treating a disease, comprising a biodegradable matrix material and at least one drug, wherein the at least one drug is released into the body and wherein the biodegradable matrix material dissolves or degrades.

2. A device according to claim 1, wherein the device comprises a ring-like, flag-like, or a plaster-like configuration.

3. A device according to claim 1, wherein the biodegradable matrix material comprises a polymeric material, a metallic material, or a combination thereof.

4. A device according to claim 1, wherein the biodegradable matrix material comprises an epoxy, polyester, acrylic, nylon, silicone, polyanhydride, polyurethane, polylactide poly(L-lactide), poly(D-lactidepoly), copolymer derived therefrom polylactide poly(L-lactide) or poly(D-lactidepoly), polycarbonate, poly(tetrafluoroethylene) (PTFE), polycaprolactone, polyethylene oxide, polyethylene glycol, poly(vinyl chloride), polylactic acid, polyglycolic acid, polypropylene oxide, poly(akylene)glycol, polyoxyethylene, sebacic acid, polyvinyl alcohol (PVA), 2-hydroxyethyl methacrylate (HEMA), polymethyl methacrylate, 1,3-bis(carboxyphenoxy)propane, phosphatidylcholine, triglyceride, polyhydroxybutyrate (PHB), polyhydroxyvalerate (PHV), poly(ethylene oxide) (PEO), poly ortho ester, poly(amino acid), polycynoacrylate, polyphophazene, polysulfone, polyamine, poly(amido amine), siloxane-based elastomer, siloxane-based elastomer comprising 3,3,3-trifluoropropyl groups, lipid, isopropyl styrene, flexible fluoropolymer, vinyl pyrrolidone, cellulose acetate dibutyrate, silicone rubber, hydroxapatite, fibrin, graphite, manganese-lithium alloy comprising from about 0.5% to about 20 wt % of lithium, or any combination thereof.

5. A device according to claim 1, wherein the biodegradable matrix material comprises a naturally occurring protein, elastin, collagen, albumin, keratin, fibronectin, silk, silk fibroin, actin, myosin, fibrinogen, thrombin, aprotinin, antithrombin III, genetically engineered protein polymer consisting of silk-like blocks, elastin-like blocks, collagen-like blocks, laminin-like blocks, fibronectin-like blocks, and the combination of silk-like and elastin-like blocks, or any combination thereof.

6. A device according to claim 1, wherein the biodegradable matrix material comprises a shape-memory effect material.

7. A device according to claim 6, wherein the biodegradable matrix material that hardens via an increase in its temperature with an energy source.

8. A device according to claim 1, wherein the device comprises different areas, with each area comprising a different drug or a different area comprising the same drug in different concentrations.

9. A device according to claim 1, wherein the at least one drug comprises Taxol®, Paxitaxel™, adenosine, Aldactone®, alteplace, amlodipine, amiodarone, anistreplase, aspirin, atenolol, atropine, abciximab, captopril, carvedilol, Celebrex®, chlorothiazide, cholestyramine, clofibrate, clopidrogel, digoxin, dipyridamole, disopyramide, dobutamine, dofetilide, dopamine, enalapril, epinephrine, felodipine, Flecainide, Furosemide™, Heparin, Hydralazine, Ibutilide, Isosorbide dinitrate, Labetalol, Lidocaine, lisinopril, Losartan, Lovastatin, Methydopa, Metoprolol, Minoxidil, nifedipine, Nimodipine, Nitropusside, Pravastatin, Procainamide, Propranolol™, protamine, simvastatin, sotalol, streptokinase, ticlodipine, urokinase, verapamil, warfarin, or any combination thereof.

10. A device according to claim 1, wherein the at least one drug is selected from the group consisting of resins, fibrates, niacin and statins.

11. A device according to claim 1, further comprising one or more particles, wherein the at least one drug is coated onto or incorporated into the one or more particles, and the particles are incorporated into the biodegradable matrix material.

12. A device according to claim 11, wherein the particles comprise iron oxide (Fe₃O₄), titanium, titanium alloy, titaniumoxide (TiO₂), manganese oxide, magnesiumoxide, palladium oxide, palladiumcobalt, ceramic, bioceramic, glass bioglass, glass-ceramic, resin, cement, hydroxyapatite, calcium sulfate, Al₂O₃, tricalcium phosphate, calcium phosphate salt, alginate, carbon, cobalt-based alloy, stainless steel-based alloy, titanium-based alloy, zirconium oxide, zirconia, aluminum-based alloy, vanadium-based alloys, molybdenum-based alloy, nickel-based alloy, iron-based alloy, zinc-based alloy, zinc phosphate, zinc polycarboxylate, or any combination thereof.

13. A device according to claim 1, further comprising a drug releasing agent.

14. A device according to claim 1, further comprising depots for storing the at least one drug, wherein the depots open as the matrix material dissolves or degrades.

15. A device according to claim 1, further comprising Zyn-Linkers.

16. A device according to claim 1, further comprising a binder.

17. A device according to claim 16, wherein binder comprises a synthetic polymer, dextran, any sugar based substance, starch, chitosan, agarose, albumin, or any combination thereof.

18. A device according to claim 11, wherein the particle size is in the range from about 40 nanometers to about 1 micrometer.

19. A device according to claim 1, further comprising one or more particles that change the contrast in a radiological imaging system.

20. A device according to claim 19, wherein the one or more particles comprise iron-oxide (Fe₃O₄), titanium, titanium-alloys, titaniumoxide (TiO₂), manganese oxide, magnesiumoxide, palladiumoxide, palladiumcobalt, ⁹⁰Y, ¹³³Xe, ^{81m}Kr, ¹¹¹In, ^{133m}In, ²⁰¹Th, or any combination thereof.

21. A device according to claim 1, wherein the device is attached to a vessel wall via mechanical expansion and clamping.

22. A device according to claim 1, wherein the device is attached to a vessel wall via glue.

23. A method to treat or prevent a disease, comprising
a. deploying an implantable device, comprising a biodegradable matrix material, and at least one drug; and
b. releasing the drug as the biodegradable matrix material dissolves or degrades over a period of time,
wherein the device is deployed into a vessel of a patient's body.

24. A method according to claim 23, wherein the method is used for treating a vascular or cardiovascular disease.

25. A method according to claim 23, wherein the disease is vascular plaque, cardiovascular plaque, or arteriosclerosis.

26. A method according to claim 23, wherein the device comprises a ring-like, flag-like, or a plaster-like configuration.

27. A method according to claim 23, wherein the at least one drug comprises a resin, fibrate, niacin, statin, Taxol®, Paxitaxel™, adenosine, Aldactone®, alteplace, amlodipine, amiodarone, anistreplase, aspirin, atenolol, atropine, abciximab, captopril, carvedilol, Celebrex®, chlorothiazide, cholestyramine, clofibrate, clopidrogel, digoxin, dipyridamole, disopyramide, dobutamine, dofetilide, dopamine, enalapril, epinephrine, felodipine, Flecainide, Furosemide™, Heparin, Hydralazine, Ibutilide, Isosorbide dinitrate, Labetalol, Lidocaine, lisinopril, Losartan, Lovastatin, Methydopa, Metoprolol, Minoxidil, nifedipine, Nimodipine, Nitropusside, Pravastatin, Procainamide, Propranolol™, protamine, simvastatin, sotalol, streptokinase, ticlodipine, urokinase, verapamil, warfarin, or any combination thereof.

28. A method according to claim 23, wherein the at least one drug comprises an anti-inflammatory agent, and wherein the method is used for treating or preventing vascular or cardiovascular disease, rheumatoid arthritis, diabetes, or Alzheimer's disease.

29. A method according to claim 23, wherein the biodegradable matrix material comprises an epoxy, polyester, acrylic, nylon, silicone, polyanhydride, polyurethane, polylactide poly(L-lactide), poly(D-lactidepoly), copolymer derived therefrom polylactide poly(L-lactide) or poly(D-lactidepoly), polycarbonate, poly(tetrafluoroethylene) (PTFE), polycaprolactone, polyethylene oxide, polyethylene glycol, poly(vinyl chloride), polylactic acid, polyglycolic acid, polypropylene oxide, poly(akylene)glycol, polyoxyethylene, sebacic acid, polyvinyl alcohol (PVA), 2-hydroxyethyl methacrylate (HEMA), polymethyl methacrylate, 1,3-bis(carboxyphenoxy)propane, phosphatidylcholine, triglyceride, polyhydroxybutyrate (PHB), polyhydroxyvalerate (PHV), poly(ethylene oxide) (PEO), poly ortho ester, poly(amino acid), polycynoacrylate, polyphophazene, polysulfone, polyamine, poly(amido amine), siloxane-based elastomer, siloxane-based elastomer comprising 3,3,3-trifluoropropyl groups, lipid, isopropyl styrene, flexible fluoropolymer, vinyl pyrrolidone, cellulose acetate dibutyrate, silicone rubber, hydroxapatite, fibrin, graphite, manganese-lithium alloy comprising from about 0.5% to about 20 wt % of lithium, or any combination thereof.

30. A method according to claim 23, wherein the at least one drug dilutes over a period of time selected from the group consisting of up to ten years, up to one year, up to six months, up to one month, up to one week, and up to one day.
